(19) United States
(12) Patent Application Publication
(10) Pub. No.: US 2017/0281256 A1
(43) Pub. Date: Oct. 5, 2017

(54) METHODS AND DEVICES FOR THERMAL SURGICAL VAPORIZATION AND INCISION OF TISSUE

(71) Applicant: Novoxel Ltd., Natan (IL)

(72) Inventors: Michael SLATKINE, Herzlia (IL); Ronen SHAVIT, Tel Aviv (IL); Raphael SHAVIT, Tel Aviv (IL)

(21) Appl. No.: 15/511,314

(22) PCT Filed: Sep. 10, 2015
(86) PCT No.: PCT/IL2015/050925

§ 371 Date: Mar. 15, 2017

Related U.S. Application Data
(63) Continuation-in-part of application No. PCT/IL2014/051103, filed on Dec. 16, 2014.
(60) Provisional application No. 62/103,746, filed on Jan. 15, 2015.
(60) Provisional application No. 62/050,244, filed on Sep. 15, 2014.
(60) Provisional application No. 61/917,435, filed on Dec. 18, 2013.

(51) Int. Cl. A61B 18/08; A61B 18/28; A61B 18/22

(52) U.S. Cl. CPC A61B 18/08; A61B 18/22; A61B 18/28; A61B 2018/0019

(57) ABSTRACT

A device for thermal incision of tissue including a tissue heating element, an oscillatory mechanism that advances the tissue heating element toward tissue and retracts the tissue heating element from tissue, a detector that detects when the tissue heating element contacts the tissue, and a heat controller that controls heating of the tissue heating element, wherein the heat controller for the tissue heating element controls heating the tissue heating element based on detecting when the tissue heating element contacts tissue. Related apparatus and methods are also described.

### RELATED APPLICATION/S

This application claims the benefit of priority from U.S. Provisional Patent Application No. 62/103,746 filed 15 Jan. 2015, U.S. Provisional Patent Application No. 62/050,244 filed 15 Sep. 2014, and International Application No. PCT/IL2014/051103 filed 16 Dec. 2014, which claims priority from U.S. Provisional Patent Application No. 61/917,435 filed 18 Dec. 2013, the contents of all of which are incorporated herein by reference in their entirety.

This application is also related to co-filed, co-pending and co-assigned PCT Patent Application titled “METHODS AND DEVICES FOR THERMAL TISSUE VAPORIZATION AND COMPRESSION” (Attorney Docket No. 63941) by Michael SLATKINE, Ronen SHAVIT, Raphael SHAVIT, the disclosure of which is incorporated herein by reference.

### FIELD AND BACKGROUND OF THE INVENTION

The present invention, in some embodiments thereof, relates to devices and methods for incising tissue using a tissue heating and/or vaporizing element and, more particularly, but not exclusively, to devices and methods for sensing when the tissue vaporizing element contacts the tissue to be cut, and optionally synchronizing heating the tissue vaporizing element mainly or even only when the tissue vaporizing element contacts the tissue.

Various techniques are known for performing incision of tissue with contact surgical probes and good hemostasis. A common technique is based on electrosurgery. Monopolar electrosurgical units (ESU) can provide precise incisions when properly controlled. However, return current to ground pads or to metallic instruments such as laparoscope tubes or metallic body implants such as dental implants may cause severe burns and results in medical complications. See for example an article titled “Preventing Patient Thermal Burns from Electrosurgical Instruments, by Anne Reed, Reprinted with permission of Infection Control Today 2013.

Monopolar ESUs are not allowed in brain surgery. On the other hand, bipolar ESU provides significant tissue damage. Although ESU units are widely used in surgery, a need for precise hemostatic contact incisions in many surgical applications has been recognized some 30 years ago.

A Shaw scalpel is an example of such a device. The Shaw scalpel is a sharp blade which can be heated by an internal electrical wire up to a temperature of 280 deg C. The controller can control the scalpel blade temperature within a narrow limit. The blade is also sharp enough to permit cold incision. A disadvantage of the Shaw scalpel is its inability to be used in endoscopic procedures as well as its relatively slow heating and cooling time. The Shaw scalpel is used as a continuous incision device, often resulting in peripheral thermal damage which depends on incision speed. Furthermore, depth of incision is not automatically controlled and varies according to user applied vertical and lateral forces. See for example an article titled “Use of the Shay scalpel in head and neck surgery”, by Willard E. Fee, published in Otolaryngol Head Neck Surg 89:515-519 (July-August) 1981.

U.S. Pat. No. 4,736,743 to Daikozono describes a medical laser probe for contact laser surgery wherein a surgical incision, for example, is made by direct and indirect laser heating of the tissue. Direct heating is achieved in the conventional manner by direct laser irradiation of the subject tissue. Indirect heating is achieved through the use of a probe tip specially coated with infrared absorbing material. The material serves to partially absorb and partially transmit the laser energy. The absorbed laser energy heats the probe tip thereby facilitating tissue vaporization when the probe is brought into contact with the tissue. The transmitted laser energy vaporizes the tissue by the conventional irradiation thereof. The tip surface is roughened prior to application of the infrared material to enhance adhesion while an optically transparent material is placed over the tip to preclude material damage or erosion during normal tip use.

Sapphire tips are probes attached to a distal end of an optical fiber. A proximal end of the optical fiber is fed with laser light, such as emitted from an Nd:YAG laser and the light is conducted along the optical fiber by total internal reflection. The optical radiation is concentrated at the tip end and is absorbed by tissue, followed by a transfer of heat from the tissue to the tip of the optical fiber, and generating a thin semitransparent layer of carbonized tissue when the optical fiber is in contact with the tissue. The combination of a high temperature distal tip of the optical fiber as well as light absorbed by carbonized tissue vaporizes the tissue and provides an incision upon moving the tip on the tissue. Such an incision is less precise than an incision obtained with a focused CO₂ laser, yet has an advantage of providing tactile feedback. Light which partially leaves the tip distal end and propagates into tissue further heats tissue beyond the coagulation thermal damage. Similarly to the Shaw scalpel described above, an incision depth obtained with a sapphire tip is not well controlled. If an operator hand moves along a curve which is not parallel to the tissue surface, the incision depth will not be homogeneous and will generally follow the hand moving curve. In addition, if hand movement is too slow, considerable thermal damage is generated, and if hand movement is too fast, tissue is not vaporized and heat damage is even larger.

Other forms of laser based contact incision of tissue utilize bare optical fibers without a sapphire probe. In such cases the distal end of the thin (mostly up to 600 micron diameter) bare optical fiber is coated by carbonized tissue upon turning on the laser, followed by enhanced optical absorption. These fibers incise tissue similarly to Sapphire probes, however the fibers are highly fragile and often melt or break after a short use. An example of a thermal surgical contact fiber which utilizes a laser as an optical energy source is the FiberTom, produced by Medilas, Dornier Medtech and described in www.dornier.com.

U.S. Pat. No. 6,383,179 to Neuberger describes a device that simultaneously incises an area and cauterizes the desired tissue. The device incorporates laser energy by some means into a mechanical scalpel so that the incised area is cauterized as well. For example, a laser source is coupled by some means to an optically transparent blade such as a diamond knife. The diamond knife is appropriately coated so that radiation only exits at desired areas. In another example, optical fibers are embedded into a sharp edge blade scalpel with means to couple to a suitable radiation source.

Published PCT patent application WO2011/013118 describes a device for vaporizing a hole in tissue, including a vaporizing element, a heating element, configured to heat the vaporizing element, and a mechanism configured to advance the vaporizing element into a specific depth in the tissue and retract the vaporizing element from the tissue within a period of time long enough for the vaporizing element to vaporize the tissue and short enough to limit diffusion of heat beyond a predetermined collateral damage distance from the hole. Related apparatus and methods are also described.

European patent application EP 1563788 describes a method of enhancing the permeability of the skin to an analytic for diagnostic purposes or to a drug for therapeutic purposes describes utilizing micro-pore and optionally sonic energy and a chemical enhancer. If selected, the sonic energy may be modulated by means of frequency modulation, amplitude modulation, phase modulation, and/or combinations thereof. Micro-pore is accomplished by (a) ablating the stratum corneum by localized rapid heating of water such that water is vaporized, thus eroding cells; (b) puncturing the stratum corneum which a micro-lancet calibrated to form a micro-pore of up to about 1000 μm in diameter; (c) ablating the stratum corneum by focusing a tightly focused beam of sonic energy onto the stratum corneum; (d) hydraulically puncturing the stratum corneum with a high-pressure jet of fluid to form a micro-pore of up to about 1000 μm in diameter; or (e) puncturing the stratum corneum with short pulses of electricity to form a micro-pore of up to about 1000 μm in diameter.

U.S. Pat. No. 5,498,258 to Hakky describes a device and method for coagulating, lasing, resecting and removing prostate and bladder tissue. The device is a laser resectoscope containing laser induced mechanical cutting. The tips of the cutting blades are coated with Teflon and Stainless Steel to prevent adherence of the lased or resected tissue. The contact laser head and cutting blades are heated by a laser beam. This allows the operator to lase and resect the targeted tissue without impairing the cellular integrity of the tissue. Consequently, the retrieved tissue is preserved for histological analysis. A method is also provided to coagulate, lase, resect and remove tissue from the prostate and bladder areas using the above mentioned laser resectoscope with laser induced heating.

U.S. Pat. No. 8,808,311 describes surgical instruments that are coupleable to or have an end effector or a disposable loading unit with an end effector, and at least one micro-electromechanical system (MEMS) device operatively connected to the surgical instrument for at least one of sensing a condition, measuring a parameter and controlling the condition and/or parameter.

U.S. Pat. No. 8,834,461 describes devices, systems and methods for the ablation of tissue. Embodiments include an ablation catheter that has an array of ablation elements attached to a deployable carrier assembly. The carrier assembly can be constrained within the lumen of a catheter, and deployed to take on an expanded condition. The carrier assembly includes multiple electrodes that are configured to ablate tissue at low power. Additional embodiments include a system that includes an interface unit for delivering one or more forms of energy to the ablation catheter.

U.S. Pat. No. 8,876,811. A flexible fiber delivers laser optical radiation to various surgical sites by mechanically bending the fiber.

The disclosures of all references mentioned above and throughout the present specification, as well as the disclosures of all references mentioned in those references, are hereby incorporated herein by reference.

### SUMMARY OF THE INVENTION

The present invention, in some embodiments thereof, relates to devices and methods for incising tissue using a tissue heating and/or vaporizing element and, more particularly, but not exclusively, to devices and methods for sensing when the tissue vaporizing element contacts the tissue to be cut, and optionally synchronizing heating the tissue vaporizing element mainly or even only when the tissue vaporizing element contacts the tissue.

According to an aspect of some embodiments of the present invention there is provided a device for thermal incision of tissue including a tissue heating element, an oscillatory mechanism that advances the tissue heating element toward tissue and retracts the tissue heating element from tissue, a detector that detects when the tissue heating element contacts the tissue, and a heat controller that controls heating of the tissue heating element, wherein the heat controller for the tissue heating element controls heating the tissue heating element based on detecting when the tissue heating element contacts tissue.

According to some embodiments of the invention, the tissue heating element includes a material selected from a group consisting of metal, and metal coated with a bio-compatible coating.

According to some embodiments of the invention, further including a laser that heats the tissue heating element, and an optical fiber that conducts output of the laser to the tissue heating element.

According to some embodiments of the invention, the tissue heating element includes a material selected from a group consisting of sapphire, metal, and metal coated with a bio-compatible coating.

According to some embodiments of the invention, the tissue heating element includes a material opaque to optical energy emitted from the laser.

According to some embodiments of the invention, further including a laser that heats the tissue heating element, and an optical fiber that conducts output of the laser to the tissue, in which a tip of the optical fiber passes heat into the tissue, thereby including the heating element.

According to some embodiments of the invention, further including an electric conducting element that heats the tissue heating element.

According to some embodiments of the invention, the tissue heating element includes an electric conducting element.

According to some embodiments of the invention, the detector for detecting when the tissue heating element contacts the tissue includes a detector that measures mechanical impedance to advancing the tissue heating element.

According to some embodiments of the invention, the oscillatory mechanism that advances the tissue heating element toward tissue and retracts the tissue heating element from tissue is arranged to advance the tissue heating element toward tissue for a distance in a range of 0-20 millimeters beyond where the detector that detects when the tissue heating element contacts the tissue detects the tissue heating element contacting the tissue.

According to some embodiments of the invention, the oscillatory mechanism that advances the tissue heating element toward tissue and retracts the tissue heating element from tissue includes an electric motor.

According to some embodiments of the invention, the detector that detects when the tissue heating element contacts the tissue includes a detector that measures current to the electric motor.

According to some embodiments of the invention, the mechanism that advances the tissue heating element toward tissue and retracts the tissue heating element from tissue includes a linear electric motor.

According to an aspect of some embodiments of the present invention there is provided a method for incising tissue including using a device for thermal incision of tissue for automatically advancing a tissue heating element toward tissue, automatically detecting when the tissue heating element contacts tissue, and automatically controlling heating the tissue heating element based on detecting when the tissue heating element contacts tissue.

According to some embodiments of the invention, further including automatically advancing the tissue heating element a desired distance measured from a point of contact with the tissue.

According to some embodiments of the invention, further including automatically retracting the tissue heating element from contact with the tissue.

According to some embodiments of the invention, further including automatically advancing and retracting the tissue heating element a plurality of times to achieve a desired depth of cumulative advance into the tissue.

According to some embodiments of the invention, further including moving the tissue heating element sideways relative to the tissue while advancing and retracting the tissue heating element a plurality of times to achieve an incision into the tissue.

According to some embodiments of the invention, the automatically detecting when the tissue heating element contacts tissue includes measuring current to an electric motor.

According to some embodiments of the invention, the automatically detecting when the tissue heating element contacts tissue includes measuring a rate of advance of the tissue heating element.

According to some embodiments of the invention, the measuring a rate of advance of the tissue heating element includes measuring advance of the tissue heating element and calculating the rate by dividing the advance by a duration of the advance.

According to some embodiments of the invention, heating the tissue heating element is controlled to start only after automatically detecting when the tissue heating element contacts tissue.

According to some embodiments of the invention, heating the tissue heating element is controlled to start a desired period of time after automatically detecting when the tissue heating element contacts tissue.

According to some embodiments of the invention, heating the tissue heating element is controlled to last for a desired period of time and then stop.

According to some embodiments of the invention, the desired period of time for heating the tissue is calculated based on an amount of heat required for evaporating a desired volume of tissue.

According to some embodiments of the invention, the amount of heat required for evaporating a desired volume of tissue is calculated based on a cross section of the tissue heating element in contact with tissue multiplied by a depth desired for a crater in a single round of advancing the tissue heating element into tissue and retracting the tissue heating element from the tissue.

According to some embodiments of the invention, the device for thermal incision of tissue includes a laser for heating the tissue heating element, and an optical fiber for conducting output of the laser to the tissue heating element, and in which the automatically controlling heating the tissue heating element includes causing the laser to produce output.

According to an aspect of some embodiments of the present invention there is provided a device for introduction of material through a tissue by vaporizing a crater in the tissue including a tissue heating element, a mechanism that advances the tissue heating element toward tissue, a detector that detects when the tissue heating element contacts the tissue, and a heat controller that controls heating of the tissue heating element, wherein the heat controller for the tissue heating element controls heating the tissue heating element based on detecting when the tissue heating element contacts tissue.

According to an aspect of some embodiments of the present invention there is provided a method for introduction of material through a tissue by vaporizing a crater in the tissue including using a device for thermal incision of tissue for automatically advancing a tissue heating element toward tissue, automatically detecting when the tissue heating element contacts tissue, and automatically controlling heating the tissue heating element based on detecting when the tissue heating element contacts tissue.

According to some embodiments of the invention, a duration of the crater remaining open to introduction of the material, prior to closure by healing, is above 1 hour.

According to some embodiments of the invention, contact time of the tissue heating element with tissue is automatically kept shorter than 10 milliseconds.

According to some embodiments of the invention, a width of a distal end of a tip of the tissue heating element is less than 150 microns.

According to some embodiments of the invention, a tip of the tissue heating element includes a material selected from a group consisting of stainless steel and titanium.

According to some embodiments of the invention, the duration is above 6 hours.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a simplified block diagram illustration of a device for incising tissue in surgical procedures according to an example embodiment of the invention;

FIG. 1B is a simplified block diagram illustration of a process of linear incision using an example embodiment of the invention;

FIGS. 1C and 1D are more detailed illustrations of line incision produced according to an example embodiment of the invention;

FIG. 1E is a simplified illustration of producing a line incision according to an example embodiment of the invention;

FIG. 1F is an illustration of a feature of a hand-piece according to another example embodiment of the invention;

FIG. 1G is a simplified block diagram illustration of a device for incising tissue in surgical procedures according to another example embodiment of the invention;

FIGS. 2A and 2B are simplified block diagram illustrations of interaction between a heated tip and tissue according to example embodiments of the invention;

FIGS. 3A and 3B are simplified block diagram illustrations of interaction between prior art surgical laser based sapphire contact tips and tissue;

FIGS. 4A and 4B are simplified block diagram illustrations of producing an incision of constant depth according to an example embodiment of the invention;

FIG. 5 is a simplified block diagram illustrations of producing an incision according to a prior art embodiment of conical sapphire tip;

FIG. 6 is a simplified cross-sectional illustration of an example embodiment of the invention;

FIG. 7 is a simplified flow chart illustration of a method for depth control of craters produced according to an example embodiment of the invention;

FIG. 8A is an oscilloscope trace of a position of an array of tips and of a driving current of a linear motor driving the array of tips in air according to an example embodiment of the invention;

FIG. 8B is an oscilloscope trace of a position of an array of tips and of a driving current of a linear motor driving the array of tips including a period of time touching impeding skin according to an example embodiment of the invention;

FIGS. 9A and 9B are cross section images depicting copper tips coated with a coating of nickel followed by gold according to another example embodiment of the invention;

FIG. 9C is a graph depicting concentration of elements as a function of distance along the copper tips and the nickel followed by gold coating of the example embodiment of FIGS. 9A and 9B;

FIG. 10 is a simplified illustration of treatment probe tips according to example embodiments of the invention;

FIG. 11 is a photograph of a histology cross section of an in vivo human skin vaporized crater produced immediately after treatment according to an example embodiment of the invention;

FIG. 12 is a simplified cross-sectional illustration of an example application of incising tissue according to an example embodiment of the invention;

FIG. 13 is a simplified cross-sectional illustration of an example application of incising tissue according to an example embodiment of the invention;

FIG. 14 is a simplified illustration of an array of tips, a hand-held device, and a list of features of the hand-held device according to an example embodiment of the invention;

FIGS. 15A, 15B and 15C are simplified illustrations of a process of thermo-mechanical ablation (TMA) according to an example embodiment of the invention;

FIG. 16 is a simplified illustration of a list of various effects produced by applying heated tips according to an example embodiment of the invention;

FIG. 17 includes nine cross section images depicting tissue treated in various treatment modes using various treatment tips according to various example embodiments of the invention;

FIG. 18 is a simplified illustration of an array of tips and a description of the array of tips according to an example embodiment of the invention.

FIG. 19 is a simplified illustration of a barcode optionally used with an array of tips and a description of further optional features associated with the array of tips according to an example embodiment of the invention;

FIG. 20 is a simplified illustration of a hand-piece a description of optional features associated with the hand-piece according to an example embodiment of the invention;

FIG. 21 is a simplified illustration of a system for thermal surgical vaporization and incision of tissue and a description of optional features associated with the system 2102 according to an example embodiment of the invention;

FIGS. 22A and 22B include cross section images depicting tissue treated in an experiment and a description of findings associated with the experiment according to an example embodiment of the invention;

FIG. 23 is a table comparing treatment according to an example embodiment of the invention, named Tixel, to treatment with a Fractional CO₂ laser;

FIG. 24 is a simplified illustration of an array of tips and a treatment system according to an example embodiment of the invention;

FIG. 25 is a simplified illustration of a hand-piece according to an example embodiment of the invention;

FIGS. 26A-26D are simplified illustrations of a hand-piece according to another example embodiment of the invention;

FIG. 27 is a simplified illustration of a semi-frontal view of a cross section of a hand-piece according to an example embodiment of the invention;

FIG. 28 is a simplified illustration of a hand-piece according to yet another example embodiment of the invention;

FIG. 29 is a simplified illustration of a tip and a description of the tips according to an example embodiment of the invention;

FIG. 30 is a simplified illustration of a tip and two tables according to an example embodiment of the invention;

FIG. 31 is an image of an array of tips according to an example embodiment of the invention;

FIG. 32A is an image of a hand-piece for thermal surgical vaporization and incision of tissue applied to calf's liver according to an example embodiment of the invention;

FIG. 32B is an image of the hand-piece of FIG. 32A applied to calf's liver according to an example embodiment of the invention;

FIG. 32C is an image of calf's liver incised with a heated linear tip array according to an example embodiment of the invention;

FIG. 32D is an image of an array of tips of FIG. 32A applied to calf's liver according to yet another example embodiment of the invention.

FIG. 33 is a simplified illustration of a tip; a tip holder and a table according to an example embodiment of the invention;

FIG. 34 is a simplified illustration of a tip; a tip holder and a table according to an example embodiment of the invention;

FIG. 35 is a simplified flow chart illustration of a method for incising tissue according to an example embodiment of the invention; and

FIG. 36 is a simplified flow chart illustration of a method for introduction of material through a tissue by vaporizing a crater in the tissue according to an example embodiment of the invention.

### DESCRIPTION OF SPECIFIC EMBODIMENTS OF THE INVENTION

The present invention, in some embodiments thereof, relates to devices and methods for incising tissue using a tissue vaporizing element and, more particularly, but not exclusively, to devices and methods for sensing when the tissue vaporizing element contacts the tissue to be cut, and optionally synchronizing heating the tissue vaporizing element mainly or even only when the tissue vaporizing element contacts the tissue.

Overview

An aspect of some embodiments of the invention involves automatically advancing a heating and/or vaporizing element toward tissue, determining when the vaporizing element is in contact with the tissue, heating the vaporizing element so as to vaporize tissue in contact with the vaporizing element, and automatically retracting the vaporizing element from contact with the tissue.

The example of tissue vaporizing is used herein as an example, but other effects of heating can be used to assist in tissue incision, such as, by way of some non-limiting examples: vaporizing tissue; liquefying tissue; causing connective tissue to detach; causing tissue cells to explode.

The term “vaporizing element” in all its grammatical forms is used throughout the present specification and claims interchangeably with a term “heating element” and its corresponding grammatical forms.

In some embodiments, the above effects on tissue are optionally achieved based on temperature settings, controlling a rate of temperature increase, controlling duration of heating, and so on.

An aspect of some embodiments of the invention involves how to sense when the vaporizing element is in contact with the tissue. In some embodiments, it is an electric motor, optionally a linear motor, advancing the vaporizing element. When the vaporizing element contacts tissue, the advancing requires advancing against an increased opposition, which, in some embodiments, is optionally sensed. In some embodiments, the increased opposition to movement optionally slows down the advance, and the slowing down is optionally sensed.

An aspect of some embodiments of the invention involves determining by how much to advance the vaporizing element, and relative to what point in time or space. In some embodiments, the distance of advancing is determined relative to a point of contact of the vaporizing element with tissue. In some embodiments, the distance of advancing determines a depth of a vaporized crater in tissue produced by the advancing of the vaporizing element.

An aspect of some embodiments of the invention involves making repeated advances of the vaporizing element. In some embodiments, the repeated advances are made at one location of the tissue, optionally vaporizing a deeper crater in tissue than a single advance. In some embodiments, a device from which the vaporizing element advances is held in place, and the advance of the vaporizing element is optionally measured from a first point where the vaporizing element contacts tissue, so that a cumulative depth of the vaporizing of tissue is optionally measured. In some embodiments, a desired depth of vaporizing tissue is entered as input to a control unit, and the vaporizing element is optionally advanced, once or repeatedly, until the depth of vaporizing the tissue reaches the desired depth.

An aspect of some embodiments of the invention involves making repeated advances of the vaporizing element while also moving the vaporizing element sideways. In some embodiments such repeated cratering of tissue optionally produces an incision in the tissue. In some embodiments a depth of the incision is measured or controlled as described above.

An aspect of some embodiments of the invention involves determining when to heat the vaporizing element. In some embodiments the vaporizing element is heated only when in contact with tissue, optionally determined as described above.

An aspect of some embodiments of the invention involves determining how much heat to apply to the vaporizing element in a single pulse of advancing and retracting. In some embodiments the amount of heat is calculated based on an amount of heat required for evaporating a desired volume of tissue. In some embodiments, the desired volume of tissue is calculated based on a cross section of the vaporizing element in contact with tissue multiplied by a depth desired for a crater in the single pulse. In some embodiments, the amount of heat is calculated is calculated to be greater than amount of heat required for evaporating a desired volume of tissue, to allow for vaporized tissue presenting a somewhat insulating barrier to heat conduction from the vaporizing element to the tissue. In some embodiments, the amount of heat is calculated is calculated to be greater than amount of heat required for evaporating a desired volume of tissue, to allow for heat losses in the application.

An aspect of some embodiments of the invention involves determining how to heat the vaporizing element. In some embodiments, the heating is performed by piping laser energy along a fiber optic light guide, such that the laser energy heats a vaporizing element at the end of the fiber optic light guide. Such a vaporizing element is described in more detail below. Some non-limiting examples of materials used to manufacture such a vaporizing element include: sapphire, metal, metal coated with a bio-compatible coating, and so on.

In some embodiments, the heating is performed by piping laser energy along a fiber optic light guide, such that the laser energy heats the tissue when such heating is desired. In such embodiments there is actually no vaporizing element. However, a person skilled in the art will understand that it is still possible to sense when a fiber optic is advanced toward tissue and makes contact with the tissue, so that embodiments described above which utilize sensing when the vaporizing element makes contact with tissue can be understood to mean that the fiber optic makes contact with the tissue.

In some embodiments the heating is performed by electric heating of an electric conducting element or an electric conducting foil at the vaporizing element. In some embodiments the electric conducting element or foil is the vaporizing element itself.

An aspect of some embodiments of the invention involves dimensions of, and/or a size of, and/or a shape of, the vaporizing element. In some embodiment, the size is designed to approximately correspond to a size of a hole or crater which is desired to be made in the tissue. In some embodiment, the size is designed to approximately correspond to a width of an incision which is desired to be made in the tissue, by repeatedly making holes side-by-side in the tissue. In some embodiments the shape of the vaporizing element is produced so as to facilitate making the desired hole or incision. By way of a non-limiting example, in some embodiment the vaporizing element is optionally shaped as an elongated element, facilitating making a linear incision. By way of another non-limiting example, in some embodiment the vaporizing element is optionally shaped as a linear array of vaporizing elements, also facilitating making a linear incision.

An aspect of some embodiments of the invention involves a material selected for making the vaporizing element. In some embodiment, the material is selected to have a high heat conductance. In some embodiment, the material is selected to have a heat capacity in a specific range, so as to contain enough heat, when heated, to vaporize a desired amount of tissue, corresponding to making a hole or incision of a desired depth in the tissue, and/or so as to lose heat rapidly when not heated, so as to minimize possible damage from having a hot vaporizing element near tissue.

An aspect of some embodiments of the invention involves coating the vaporizing element. In such embodiments one or more coating materials, optionally in one or more layers, coat a first material which makes up a core of the vaporizing element. In some embodiments the coating prevents the core material from coming in touch with tissue, especially if the core material is not considered safe to contact living tissue, when the core material is cold and/or when the core material is heated to a tissue-vaporizing temperature.

In some embodiments, the above effects on tissue are optionally achieved based on temperature settings, rate of temperature rise settings, and so on.

Some major disadvantages of the current laser based thermal contact probes are:

A) Laser radiation leaks into tissue from a sapphire probe or a fiber distal end. The leak results in deeper coagulation and less bleeding. However, the incision is less precise with a large peripheral thermally damaged zone resulting in slower healing. Also carbonization of tissue is common. It is noted that in some embodiments of the invention a heated tip for surgery is optionally opaque, in some cases a metallic coating over a fiber optic, which potentially prevents laser radiation leakage.

B) Lasers used, mainly Nd:YAG or diode lasers, are high power lasers, whose power in key surgical applications may exceed 30 Watts, and some lasers such as used in dentistry may use 5-10 Watts Such lasers are relatively large and expensive. Moreover, such lasers, and even 5W lasers, are subject to high class, mostly class IV, medical regulations, and require stringent safety precautions since they directly interact with tissue. As a result such lasers are in any case very expensive. In addition, operating room staff has to use uncomfortable safety glasses. It is noted that in some embodiments of the invention the tip has a low thermal capacity, such as, by way of a non-limiting example having a thin hollow metallic tip, enabling use of relatively lower power lasers, by way of a non-limiting example lasers with average power of ˜1-˜10 Watts.

It is also noted that in some embodiments of the invention a pulsed laser may optionally be used, optionally synchronized with tip contact with tissue. Such embodiments potentially use less power.

C) Incision depth is not well controlled.

D) Incision depth is not constant and controlled since operator hand may move at a varying speed and change vertical and lateral incising force and dwell time on tissue.

The present invention, in some embodiments thereof, relates to surgical methods and devices, and, more particularly, but not exclusively, to methods and devices for incision and vaporization of tissue in surgical procedures, and even more particularly, but not exclusively, to methods and devices for microsurgery such as in neurosurgery, ear-nose-throat (ENT) surgery, dentistry, delivery of drugs through tissue, and laparoscopy, among others.

The term “tissue” in all its grammatical forms is used throughout the present specification and claims interchangeably with the term “skin” and its corresponding grammatical forms. Various implementations and embodiments of the invention which are described with reference to treating tissue are intended to apply also to treating skin.

The term “crater” in all its grammatical forms is used throughout the present specification and claims interchangeably with the term “depression” and its corresponding grammatical forms. Various implementation and embodiments of the invention which are described as producing craters in tissue are intended to apply also to producing depressions in tissue.

It is one purpose of embodiments of the current invention to overcome disadvantages of prior art.

It is one purpose of embodiments of the current invention to control a depth of vaporization of tissue with high temperature tips.

It is one purpose of embodiments of the current invention to improve post treatment condition of patients.

An aspect of some embodiments of the invention involves using a tip of a heated rod, to produce an incision of tissue with minimal collateral damage and without damaging underlying tissue.

In some embodiments, a distal end of the tip is mostly metallic.

In some embodiments, a distal end of the tip is opaque.

In some embodiments, heated temperatures of the tip are between 100 and 850 degrees Celsius.

In some embodiments a vaporizing element, such as a vaporizing rod, has a distal end shaped into specific truncated shapes, adapted to supply a large amount of heat in a short amount of time to vaporize tissue, while in some embodiments also avoiding charring of the tissue.

In some embodiments, holes, grooves, craters and/or indentations are produced in the tissue.

In some embodiments conical or pyramidal vaporizing tips are used to incise tissue, potentially reducing or eliminating carbonization.

In some embodiments the incising is performed to a constant depth, regardless of speed of application and/or shaking.

An aspect of some embodiments of the invention is to accurately vaporize tissue without causing damage to underlying delicate tissue or metallic parts such as, by way of a non-limiting example, implants such as dental implants.

An aspect of some embodiments of the invention involves detecting when a surgical distal tip comes in touch with skin.

In some embodiments the detection is performed by sensing mechanical resistance of the skin to the tips pushing against it. Detecting when tip(s) come in touch with skin is meaningful when aiming for a specific depth and/or shape of the crater or incision in the surgical site.

In some embodiments detection is performed by measuring a speed of advancement of the tip(s), and detecting when the tip(s) movement is slowed by the tissue.

In some embodiments detection is performed by measuring electric parameters such as current or voltage or pulse width (under Pulse Width Modulation) required to advance the tip(s). When the tip(s) come into contact with tissue the electric parameters required to maintain the advance are changed, and contact with the tissue is optionally detected.

Another aspect of some embodiments of the current invention is vibrating the incision tip toward tissue and back out in order to generate very short contact duration with tissue, resulting in a potentially clean vaporization of an incision and/or a crater.

Another aspect of some embodiments of the invention is to vibrate the tip toward tissue and back out at a high frequency, resulting in overlap of vaporized craters and a clean incision.

Another aspect of some embodiments of the invention is to vibrate the incision tip toward tissue and back out at a high frequency to practically allow a user to stay a constant distance above tissue surface, the frequency being higher than a user response speed.

Another aspect of some embodiments of the invention involves vibrating the incision tip toward and back out of contact with tissue at a frequency high enough to enable automatically producing a constant depth incision by utilizing a tissue contact sensing mechanism.

Another aspect of some embodiments of the current invention involves using of a train of pulsed laser radiation delivered through an optical waveguide or fiber toward a vaporizing/incising tip in synchronization with an advance of the tip and its contact with tissue.

In some embodiments the laser radiation through the optical waveguide or fiber is deactivated in synchronization with fiber retraction. The synchronization potentially enables generation of extremely precise and safe surgical incisions and/or reduces thermal damage and/or reduces carbonization.

Another aspect of some embodiments of the current invention is a utilization of opaque, metallic hollow tips having a short thermal relaxation time in order to rapidly heat the tip up to temperatures as high as 100-850 deg C. and deliver most of the energy to the tissue within a short time duration.

Another aspect of some embodiments of the invention is to perform extremely precise incision or vaporization of tissue in dentistry and oral maxillofacial surgery, neurosurgery, ENT, endoscopy, GYN including laparoscopy, spinal surgery, and general surgery.

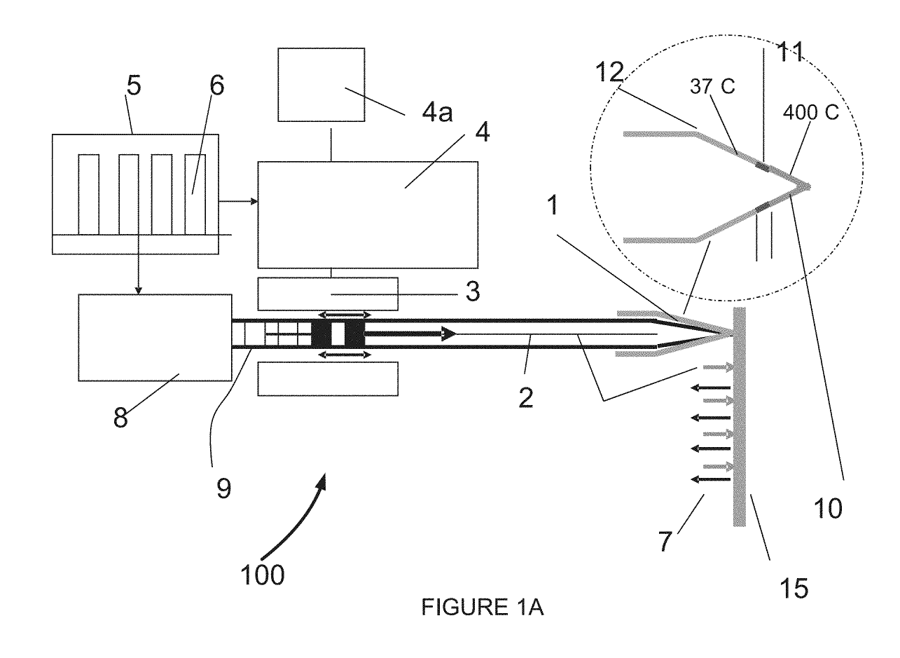

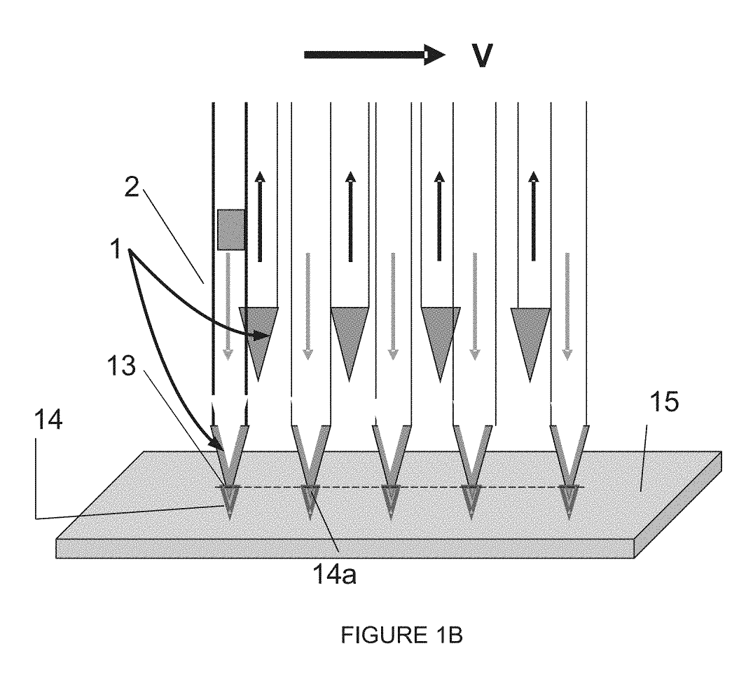

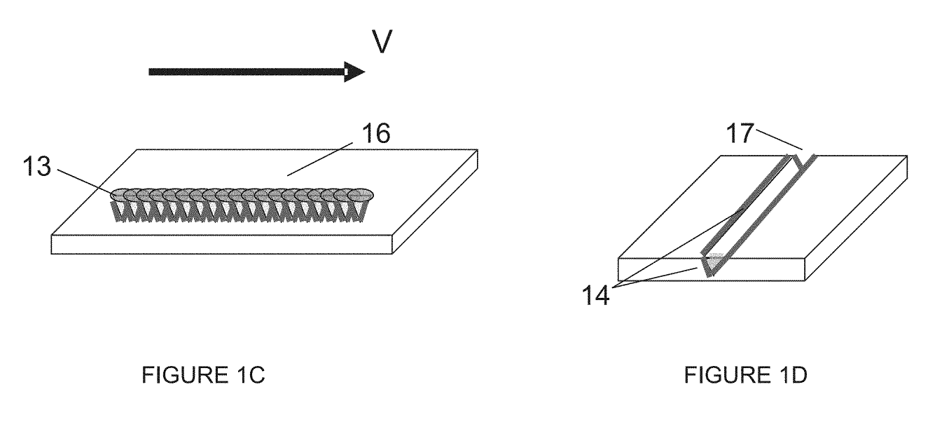

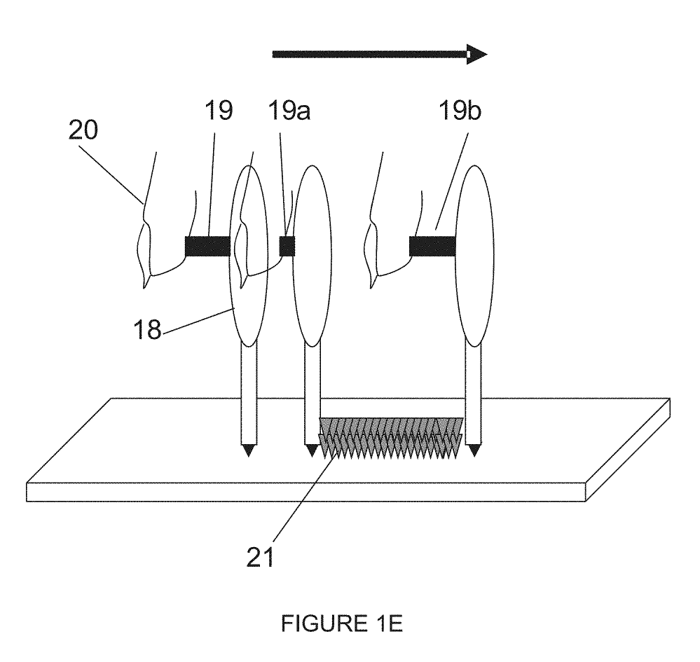

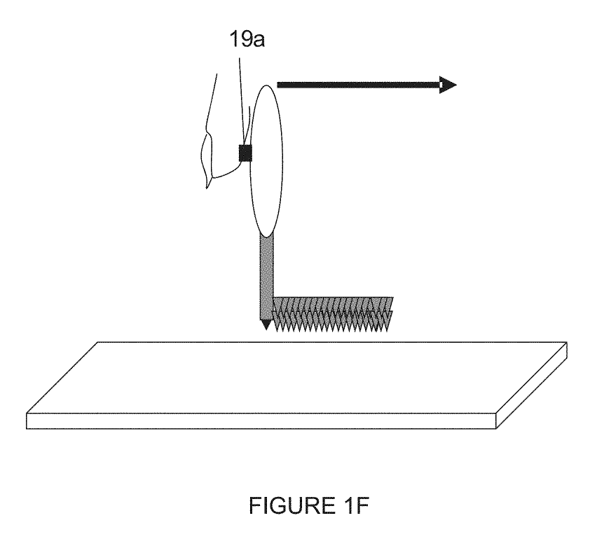

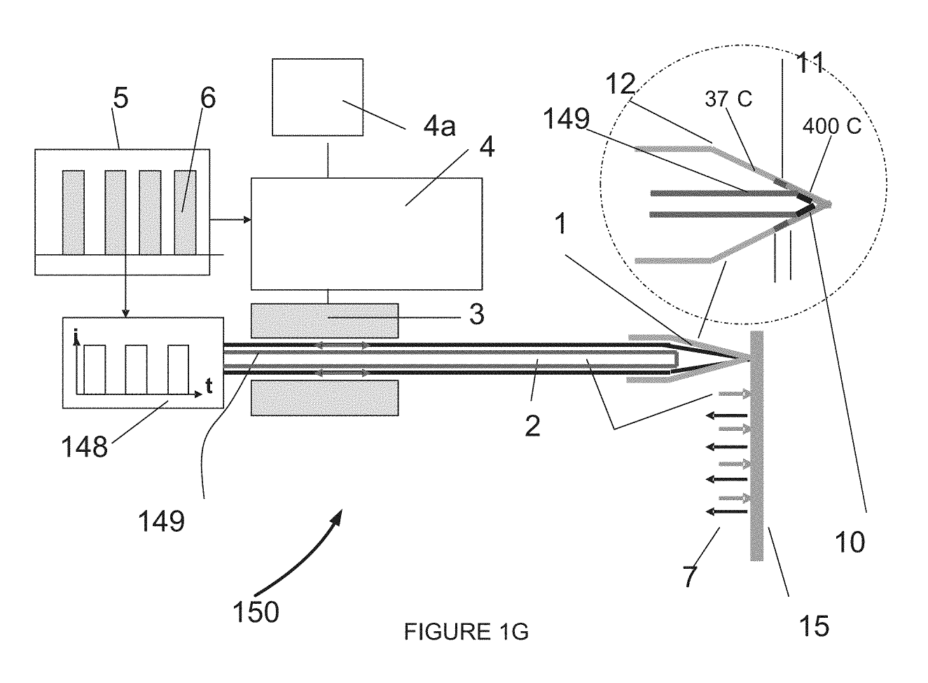

Reference is now made to FIG. 1A, which is a simplified block diagram illustration of a device 100 for incising tissue in surgical procedures according to an example embodiment of the invention.

FIG. 1A depicts a hollow metallic tip 1 attached to a distal end of an optical waveguide or fiber 2. Although other tips may be used, FIG. 1A describes an application which specifically utilizes hollow tips. The hollow metallic tip 1 may optionally have different shapes such as conical or chisel or spherical or cylindrical or pyramidal. The tip material may have a high thermal conductivity such as 150-400 W/m degrees Celsius (such as that of tungsten or copper), and may have low thermal conductivity such as lower than 30 W/m deg C. (such as stainless steel or titanium), in various applications as will be explained below.

In some example applications, high thermal conductivity is desired. The metallic tip 1 is opaque. The optical waveguide 2 is optionally attached to a motor 4, optionally by an attaching element 3. A motor 4 (which may be linear or rotary) is capable of linearly moving the fiber 2 and tip 1 toward and back from a tissue surface 15. Motion velocity as well as position and accelerations are potentially accurately known (based on position accuracy of 5-30 microns) and a motor controller 4a optionally senses contact between the tip 1 and the tissue 15, optionally based on resistance of the tissue 15 to the tip 1. Based on sensing tissue contact, the controller 4a optionally controls motor 4 speed, acceleration and position, resulting in potentially accurate control of tip 1 contact duration t with tissue as well as accurate measurement and/or control of a depth of contact. A laser 8, optionally a diode laser, emits a pulsed beam 9 which propagates along the fiber or optical waveguide 2 toward the hollow tip 1.

A master clock 5 optionally generates electrical pulses 6 which are optionally fed to one or both of the both laser diode 8 and the motor 4, resulting in synchronization between fiber motion and beam emission from the laser 8. The electric pulses result in an oscillatory motion 7 of the tip 1 whereby tip is optionally heated upon contact with tissue and optionally not heated upon retraction. The short contact duration potentially ensures a high tip temperature such as 400-850 degrees Celsius, which potentially enables clean and precise vaporization, regardless of surgeon hand speed. Moreover, since advance of incision tip is measured relative to sensing tissue contact, a depth of incision is potentially constant regardless of surgeon vertical hand movements (even hand shaking) as well as potentially independent of tissue surface curvature.

In some embodiments the optionally controlled depth is in a range of 0-20 millimeters.

In some embodiments the optionally controlled depth is controlled by measuring a distance the tip of the tissue heating element advances after detecting contact of the tip with tissue.

The optionally controlled depth is potentially beneficial in delicate surgical procedures such as opening tissue which covers a dental implant, or such as the vaporization of small lesions on vocal cords or linear incision of fallopian tube wall or vaporization of a brain lesion.

Typical parameters for a surgical hand-piece used in general surgery or dentistry include, by way of a non-limiting example:

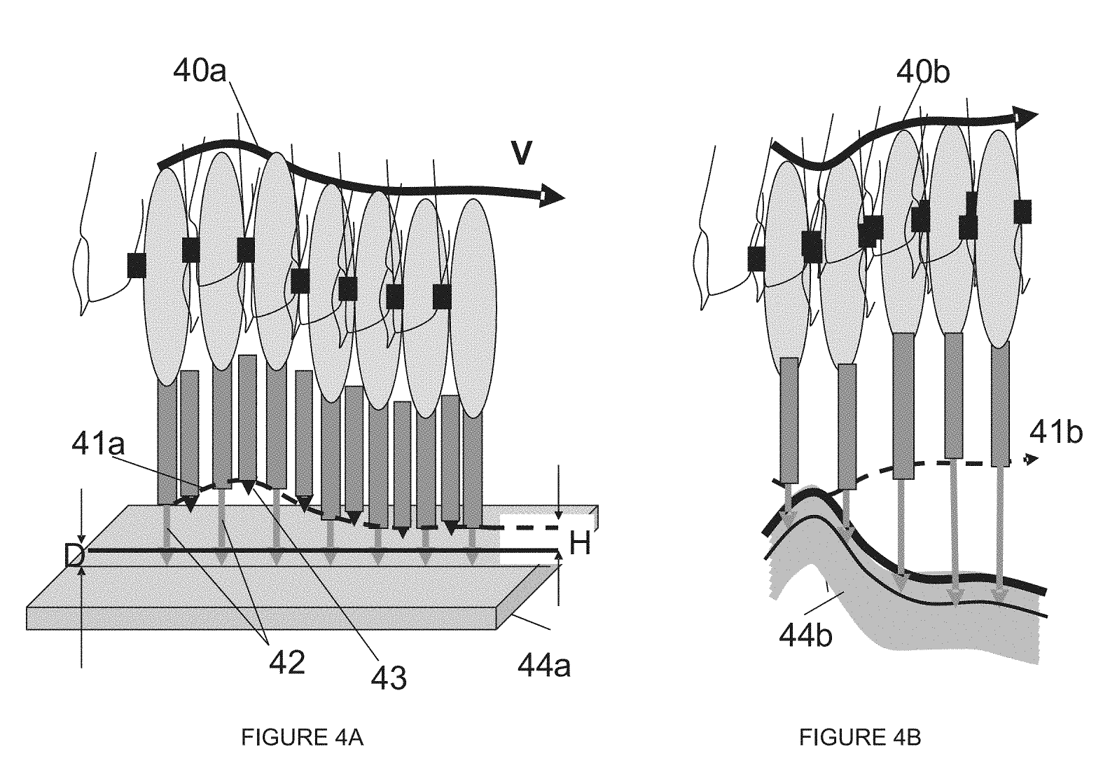

A laser power level of 5-15 Watts; an optical fiber diameter of 400-1000 microns; a tip shape: conical or chisel with tapering half angle ˜10-20 Deg (Full angle 20-40 deg); oscillatory motion amplitude 1-10 mm (optionally depends also on tissue curvature, as described below with reference to FIG. 4B; and oscillation frequency 20-100 Hz, depending on incision depth and speed. In some embodiments the oscillation frequency depends on incision speed—when a user's hand moves at high speed, for a rapid incision, the frequency is increased and prevents gaps between craters. In some embodiments the oscillation frequency depends on desired depth—optionally preventing overlap between craters so as not to produce a deep crater by advancing twice at a same location, thereby deepening the crater. Tip material: tungsten, which is biocompatible and has a high thermal conductivity (˜150 W/m deg C.). Tip wall thickness ˜100-200 microns. Distal tip temperature while heated: 400-850 deg C. Hand-piece length 100-200 mm.

In some embodiments of the invention, the fiber 2 section between the laser 8 and the attaching mechanism 3 is flexible, potentially allowing the location of the laser and its power supply to be distant from the surgical hand-piece 18, which includes the motor/controller, the distal section of the fiber 2, and the incision tip 1. In such an example embodiment case the high temperature tip 1 is distant from an optical heating source.

FIG. 1A also presents a more detailed enlargement of the distal tip 1. The walls of the hollow tip 1 are optionally shaped as a cone in its distal section, and as a cylindrical section in its proximal section, in order to enable firm attachment to the optical waveguide 2. Dimensions of the tip walls are optionally selected (as shown in calculations below) to ensure a high temperature distal end 10 for tissue incision while maintaining approximately body temperature approximately 37 degrees Celsius at a location 12 approximately 300 micron proximally (backward). A short section 11 whereby temperature drops from approximately 400-850 deg C. to approximately 37 Deg C. is approximately 300 micron long.

Reference is now made to FIG. 1B, which is a simplified block diagram illustration of a process of linear incision using an example embodiment of the invention.

FIG. 1B depicts a series of craters 13 produced by a pulsed train of advancing and retracting vaporizing tips 1 while moving a surgical hand-piece with a velocity V. Synchronization of tissue ablation with laser pulses is shown, as well as a lapse of optical heating resulting in a cold tip during a retraction phase. A tip distal end 14a optionally vaporizes craters with very shallow collateral thermal damage 14. When the velocity V is high, potentially only a train of craters is produced, optionally without creating a continuous incision. Such a train of craters is potentially useful in vaporization of thin lesions such as on vocal cords. An advantage of such embodiments is that although a velocity V may be high, tissue vaporization occurs and is clean and homogeneous. This is in contrast to use of state of the art sapphire tips where high speed, faster than energy delivery speed, entails tearing of tissue or thermal damage.

Reference is now made to FIGS. 1C and 1D, which are more detailed illustrations of line incision produced according to an example embodiment of the invention.

FIG. 1C shows a tissue incision created by a tip such as the tip of the example embodiment of FIG. 1A, where the velocity V is adjusted according to a pulsation rate. The adjustment causes an optional slight overlap between the craters 13, and produces a char free incision 16 of constant depth.

FIG. 1D shows another view of the incision of FIG. 1C. It is noted that if velocity V is slower, an overlap of craters may optionally produce a deeper crater, and still char free.

In some embodiments a surgeon may optionally notice the deeper crater, and optionally increase V.

Reference is now made to FIG. 1E, which is a simplified illustration of producing a line incision according to an example embodiment of the invention.

FIG. 1E depicts a simplified illustration of how a surgeon operates an example embodiment of an incision hand-piece 18. The surgeon optionally places the hand-piece 18 on tissue while a tip of the hand-piece 18 is cold. Upon activating the hand-piece 18, optionally by pressing a switch 19 with his finger 20, the hand-piece 18 starts the incision process and creates an incision 21. When the surgeon wants to stop, the surgeon optionally depresses the switch 19. The switch 19 may optionally be a foot-switch.

Reference is now made to FIG. 1F, which is an illustration of a feature of a hand-piece according to another example embodiment of the invention.

FIG. 1F intends to depict that when the tip of the hand-piece is not touching tissue, although a switch 19a may be activated, the tips are cold and situation is safe.

Reference is now made to FIG. 1G, which is a simplified block diagram illustration of a device 150 for incising tissue in surgical procedures according to another example embodiment of the invention.

FIG. 1G shows an embodiment of the invention in which the laser heater is replaced by a current generator 148 which is synchronized with the motor motion. Heating energy is delivered through a wire 149 also shown in the enlarged section of the figure. The electrical wire 149 is thermally coupled to the tip and in some embodiments the treatment tip is optionally coated with an oxidized layer which doesn't conduct electricity.

Other ways of heating the treatment tip, not shown in FIG. 1G, include generation of eddy currents in the tip with an oscillating magnetic field (induction heating) or ultrasound heating.

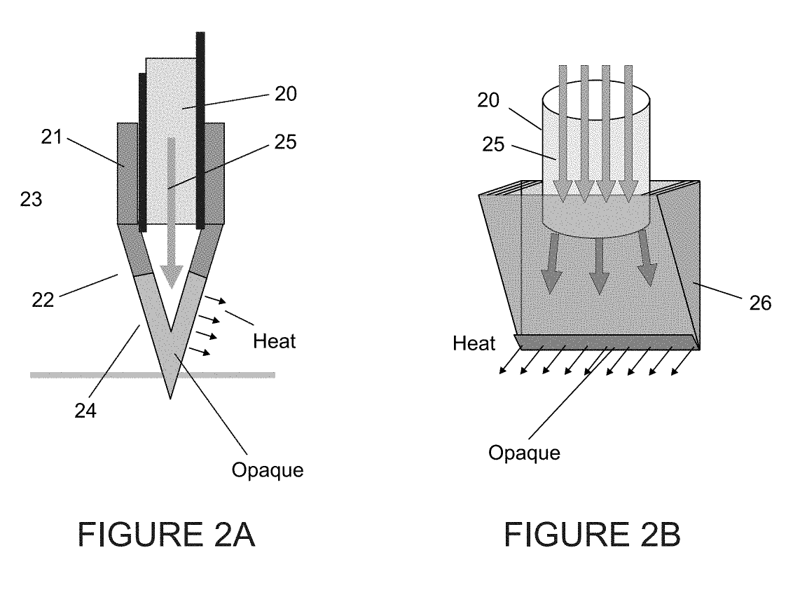

Reference is now made to FIGS. 2A and 2B, which are simplified block diagram illustrations of interaction between a heated tip and tissue according to example embodiments of the invention.

FIG. 2A presents in more detail an example conical shaped tip 26 with a heating light beam 25. Some example features in FIG. 2A are an opaque tip and producing vaporizing energy by transfer of heat from a high temperature distal end 24 of the tip to tissue.

FIG. 2B presents in more detail an example chisel shaped tip with the heating light beam 25. Some example features in FIG. 2B are an opaque tip producing vaporizing energy by transfer of heat from a high temperature distal end of the tip 26 to tissue.

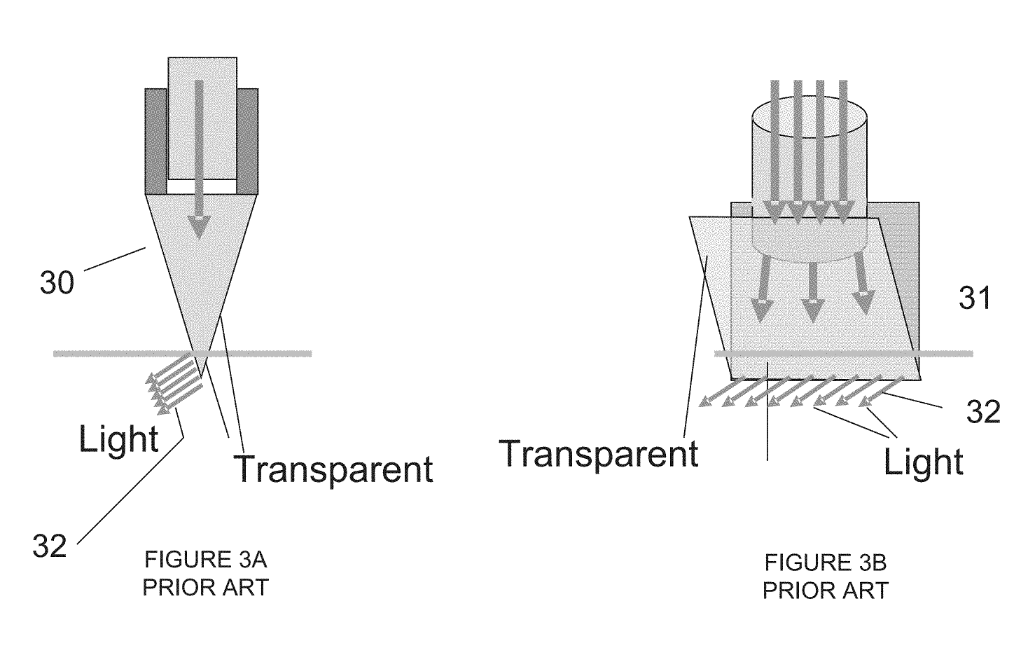

Reference is now made to FIGS. 3A and 3B, which are simplified block diagram illustrations of interaction between prior art surgical laser based sapphire contact tips and tissue;

FIGS. 3A and 3B present a tissue interaction of sapphire or fiber tips of similar shape to sapphire tips. In addition to heat transfer due to tissue carbonization, light propagates into tissue and participates in a heating process.

Reference is now made to FIGS. 4A and 4B, which are simplified block diagram illustrations of producing an incision of constant depth according to an example embodiment of the invention.

Curve 40a in FIG. 4A presents an example of an irregular movement of a surgeon's hand over tissue at a height H(t) at velocity V. H may vary over time, for example between 1-4 mm. The irregularity may be caused by momentary hand trembling or distraction or poor visibility. As a result a distal end of the hand-piece (the hand-piece excluding a tip) follows a curve 41a. While activating the hand-piece, since tip and fiber 42 optionally advance until tissue contact is established and optionally sensed by a motor controller, vaporization of depth D will occur and be identical along the incision line.

FIG. 4B also shows a similar effect, in an example where tissue surface is not flat but curved. Once again, incision is char free and of constant depth.

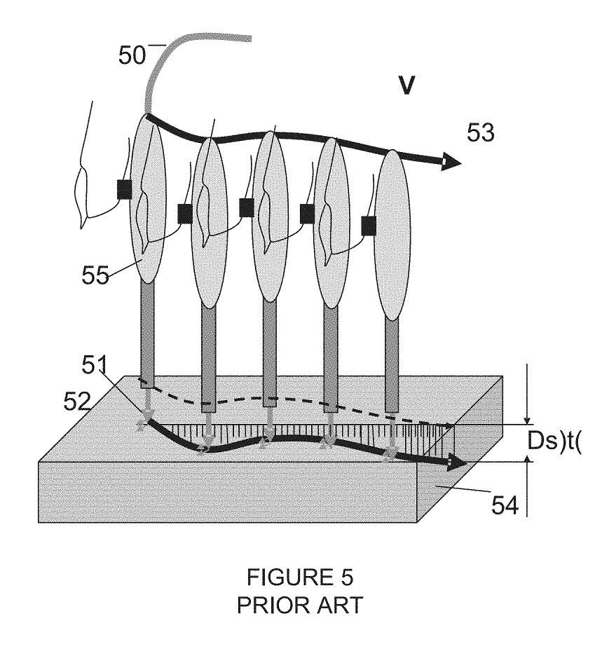

Reference is now made to FIG. 5, which is a simplified block diagram illustrations of producing an incision according to prior art embodiment of conical Sapphire tip.

The irregularity of surgeon hand movement may be translated into an irregular incision of depth D (t). It is noted that using the example embodiment method illustrated in FIGS. 4A and 4B can potentially correct the irregular incision depth of a sapphire-tipped surgical instrument to have a constant incision depth.

The following table (Table 1) lists some potential differences between features of several types of contact surgical units and an example embodiment of the invention:

| | | | Prior art Laser | An example |
|---|---|---|---|---|
| | Prior art | Prior art | based with | embodiment |
| Feature | Bipolar | Monopolar | sapphire/fiber tips | of the invention |
| Crater wall | Carbonization | Minimal | Carbonization | Excellent- |
| carbonization | | carbonization | | no/minimal |
| | | | | carbonization |
| incision depth and | Not | Less | Less | Excellent- |
| size | homogeneous | homogeneous | homogeneous | Highly |
| | | | | homogeneous |
| Energy delivery | Heat and | Electrical | Heat and light. | Heat to a |
| beyond crater/ | electrical | current- | Over 150 micron. | distance |
| incision walls | current. | very risky in | | of ~30-80 microns. |
| | Potential deeper | some | | |
| | thermal damage. | procedures | | |
| Ability to incise | Problematic | Very bad | Less, due to | Very good |
| tissue covering | thermal damage. | | thermal damage | |
| metallic implants | | | | |
| Clinical effect of | Irrelevant | Irrelevant | YES. Tip is | Potentially |
| optical radiation | | | semitransparent. | NO. Tip is opaque |
| | | | | in some |
| | | | | embodiments |
| Mechanical skin | NO | NO | NO | YES |
| contact sensing | | | | |
| and control | | | | |
| Vertical oscillatory | NO | NO | NO | YES |
| motion | | | | |
| Synchronization | NO | NO | NO | YES |
| between | | | | |
| light/laser/heat | | | | |
| pulse and tip | | | | |
| movement | | | | |
| Subject to | Irrelevant | Irrelevant | YES | NO |
| clinical safety | | | | |
| laser regulations | | | | |
| High frequency | Excellent | Excellent | Limited | Excellent |
| pulsed operation | | | | |

It is noted that not all invention features are applied in each surgical incision. For example, if oscillation frequency is high, such as 30 Hz or somewhat higher, a surgeon may feel as if the surgical hand-piece is in contact with tissue.

In another example, the heating may operate continuously and the tip will behave as a regular hot knife, optionally with a controlled depth.

An Example Embodiment of a Surgical Incision:

A surgeon wants to expose a dental implant by creating an incision of 10 mm length, 2 mm depth and 200 micron wide. Incisions along the same line are optionally repeated layer by layer. Assume each layer is 100 micron deep. As a result the surgeon will have to repeat the incision process 20 times (20 passes).

A volume of each incised (vaporized) layer is 0.2×10×0.1 mm³=0.2 mm³. The energy required to vaporize 1 mm³ of tissue is approximately 3 joule/mm³. As a result, the energy necessary to vaporize a single incision layer is 0.6 joule. If a duration of an implant exposure step is 10 second, the duration of each incision layer is 10/20=0.5 sec. As a result, the power level necessary to incise a single tissue layer within 0.5 second is 0.6/0.5 joule/sec=1.2 Watts. As a result we see that a small inexpensive industrial diode laser of 5-10 Watts can easily provide the power for making such an incision and that the incision speed may potentially be much faster.

Based on an incision width of 200 micron, a 200 micron wide distal tip end may be used and heated. Assuming a chisel tip, the number of craters along the incision line may be 10 mm/0.2 mm=50 craters. The vaporization duration of each crater may be 0.5 sec/50=10 millisec. The 10 millisec duration may be divided into two steps, 5 millisec for tissue contact and vaporization and 5 millisec for non contact motion (retraction and back to tissue). It is noted that a 5 Watt laser will use a vaporization time of 5 millisec.

Desired features for the tip are next evaluated. Assume utilization of a hollow tungsten tip with 300 micron wall thickness and a chisel shape. In order to achieve crater vaporization within 5 millisec, the treatment tip thermal relaxation time should be less than 5 millisec for a distance of L=300 micron. The thermal relaxation time Tr of a material is given by Tr ˜0.5 (cp/K) L̂2 whereby ρ=density, c=specific heat capacity, K=thermal conductivity. In the case of Tungsten, K=170 W/msec, c=0.13 j/gr, ρ=19 gr/cm³. As a result Tr ˜0.6 millisec for L=300 micron. In the case of a 1 mm size tip, Tr ˜9×0.6˜5 millisec.

In such an example, a desired width is 300 microns which is strong enough for the tip not to break or bend and potentially enables fast cooling upon retraction.

Tissue Sensing Technology for Surgery:

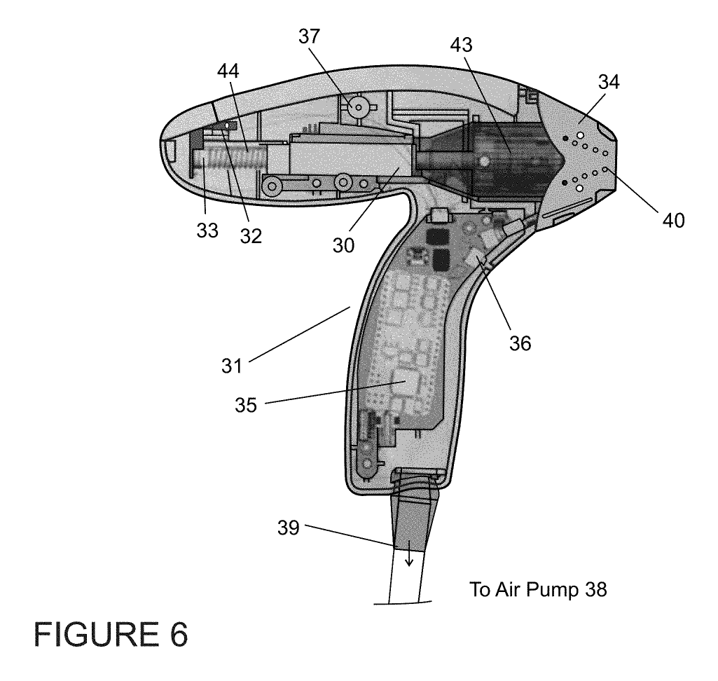

Reference is now made to FIG. 6, which is a simplified cross-sectional illustration of an example embodiment of the invention.

Referring also to FIG. 1A, motor 4 of FIG. 1A may be a rotary motor or a linear motor such as produced by Faulhaber Minimotor SA, Switzerland.

FIG. 6 presents a more detailed description of the tissue sensing technology which is generally described in FIG. 1A.

A surgical hand-piece 31 may have a “revolver” shape. The surgical hand-piece 31 may also have other shapes not depicted in FIG. 6, such as a linear pen shape as described in FIGS. 1E and 1F. A linear motor 30 is optionally located in the hand-piece 31, which may include also a position encoder 32, as depicted in FIG. 6. The position encoder 32 potentially provides a position of a rod 33 which incorporates the optical fiber/lightguide and treatment metallic probe (not shown) described in FIG. 1A, and which is driven by motor 30, relative to a reference location in the hand-piece 31. The rod 33 and fiber and distal treatment probe are pushed toward tissue (not shown) and back from tissue on which a distal cover 34 is optionally placed.

In some embodiment the distal cover 34 may be thin and elongated, for example when the hand-piece has a pen shape. The distal cover 34 may incorporate holes in order to enable suction of air into the hand-piece 31.

In some embodiments of the invention the hand-piece envelope is made of two parts.

The position encoder 32 potentially provides a 1 micron position accuracy and may be a magnetic array type encoder (such as a magnetic type encoder produced by Texas Instruments), or an optical encoder or a Hall Effect detector.

The linear motor may be operated at a constant voltage, and the force applied by it on the fiber and treatment probe may be controlled with a controller 35, optionally by modulating the width of pulses applied to the motor (Pulse width modulation—PWM).

The velocity of the rod 33, which is equal to the tip velocity, is optionally monitored by knowing the rod position. Following an advance toward tissue and upon contact with treated tissue (which may also occur after attaining a targeted incision depth) the velocity of rod 33 may be reduced if skin mechanical compliance is low. This may occur, by way of a non-limiting example, on thin tissue such as gums covering bone or when almost reaching a hard dental implant surface.

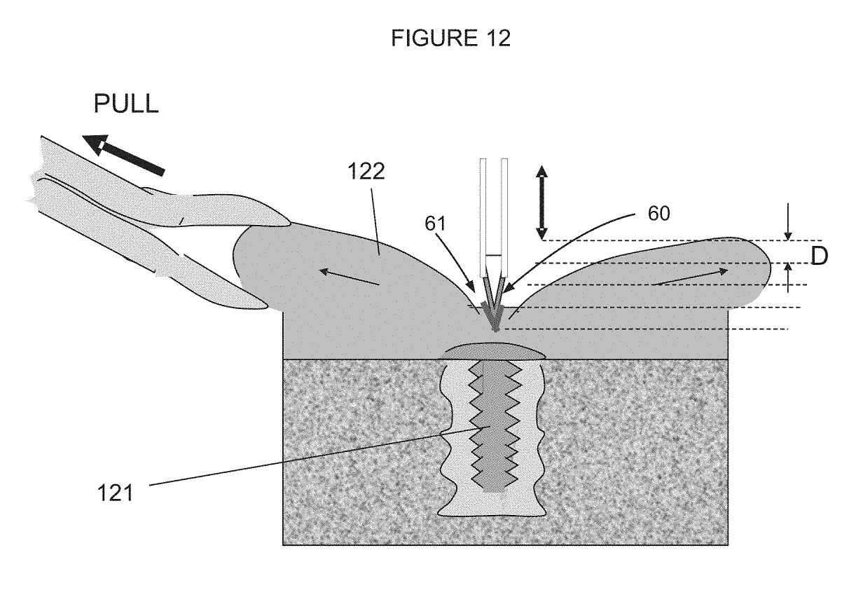

In general there is a difference between the mechanical impedance of tissue and mechanical impedance of air, resulting in good differentiation between air and tissue achieved by measuring mechanical impedance to motion of the rod/tip. In many surgical incisions, mechanical impedance of tissue is optionally further enhanced by grasping tissue with a forceps as shown in FIG. 12, which depicts an example of incising over a dental implant.

For incision depth control, various non-limiting examples of depth control strategies may be implemented.

According to a first depth-control strategy, once velocity reduction is detected, a controller optionally modifies the width of pulses applied to the motor, until velocity is restored. The rod 33 optionally continues its motion until a preselected depth is attained, whereby rod velocity is reversed and tip is retracted.

According to a second depth-control strategy the controller measures rod deceleration, and calculates rod advance distance, in order to both achieve a preset incision depth as well as a tip dwell-duration in tissue. Such a closed loop control mechanism potentially enables vaporization of craters with excellent depth accuracy (few microns) potentially regardless of tissue type (from mechanical compliance standpoint) and potentially regardless of tissue position (such as with curved tissue as depicted in FIG. 4B).

In some embodiments requirements for a stability of a surgeon's hand may be reduced or stability of treatment hand in robotic surgery may be reduced, resulting in cost reduction. Since in many treatments there is a direct relation between mechanical skin compliance and clinical side effects, such as damage due to mechanical impact or injury, a beneficial control of side effects is also potentially obtained.

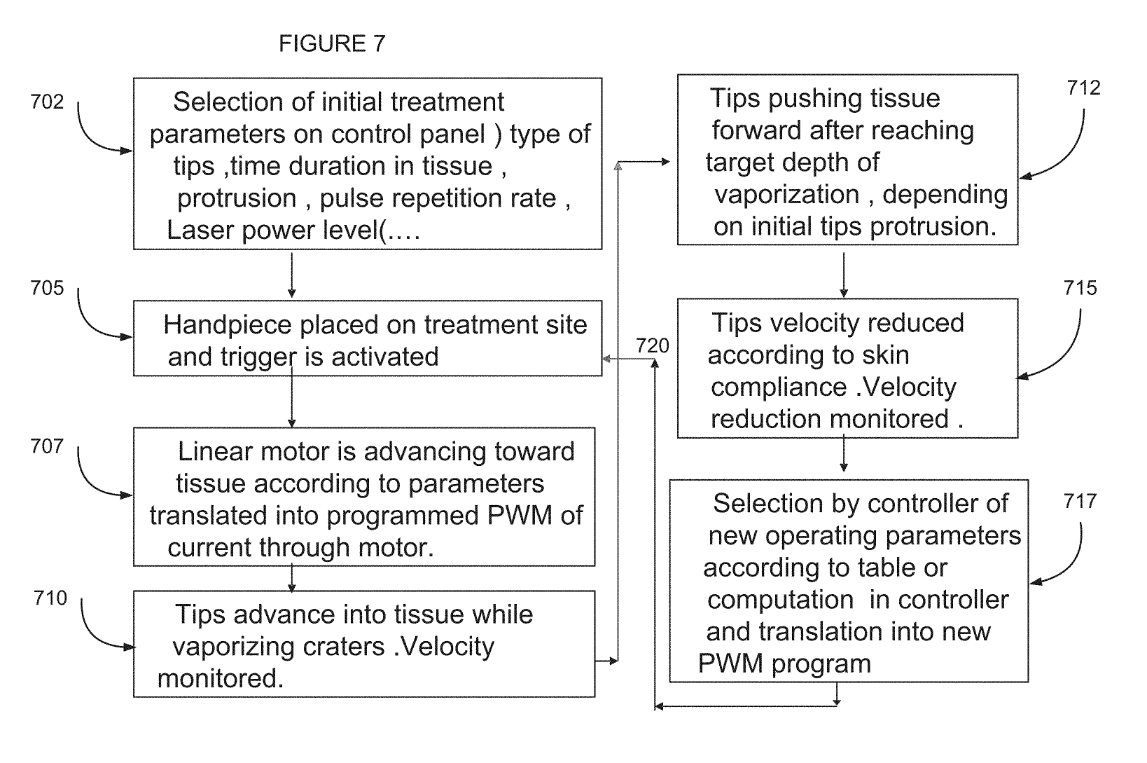

Reference is now made to FIG. 7, which is a simplified flow chart illustration of a method for depth control of craters produced according to an example embodiment of the invention.

The method depicted in FIG. 7 includes:

Providing one or more input parameters such as: type of tip, number of tips, shape of tip, dimensions of tip, duration in tissue, tip protrusion from device, pulse repetition rate, heating power level, laser power level, and so on (702);

Placing a hand-piece on a treatment site and activating a trigger (705);

Using a motor, optionally a linear motor, to advance a treatment tip or tips toward tissue, optionally at a rate based on the input parameters, optionally translated to Pulse Width Modulation (PWM) parameters for control of the motor (707);

Advancing the treatment tip(s) into the tissue, thereby producing craters, optionally while monitoring velocity or distance of advance (710);

Reaching target depth of tip(s) advance, optionally allowing the tip(s) to remain at target depth for a short time, optionally allowing tips to push against tissue (712);

Reducing tip(s) velocity, optionally based on measuring mechanical resistance of the tissue to forward motion into the tissue (715);

In some embodiments the tip touches tissue and starts to vaporize the tissue. When the tip vaporizes tissue the skin impedance to tip movement is relatively low—the skin complies with the tip motion. In some embodiments determining a vaporization depth H is dependent on energy delivered to the tip. Determining a vaporization depth H is optionally performed by determining a time duration for tip contact with tissue and/or by determining a power level of a heating unit such as a laser. Once the vaporization depth H is attained, for example 200 microns, the tissue (crater bottom) starts to resist movement of the tip since it is not being vaporized any more, only potentially heated by contact. A measurement of impedance to tip movement optionally provides data, which in some embodiments is used as a sign that vaporization has ended, and may optionally cause a decision to draw back the tip. In some embodiments, estimation of vaporization depth may optionally depend on heating parameters and time duration and depend little on surgeon hand movement, since in the embodiments the depth H depends on contact detection. In some embodiments the vaporization depth has a dependence on contact detection since without contact detection the tip might stay in contact with tissue for a longer duration in the crater, for example 30 millisec, and start heating tissue and cause peripheral thermal damage.

Optionally a controller automatically selecting new operating parameters based on measurements made, optionally translating the new operating parameters into a new (PWM) program (717); and

Optionally repeating some of 705-715 or 705-717 (720).

FIG. 7 depicts a schematic description of an example embodiment of closed loop control of depth of vaporization of craters regardless of skin compliance and precise vertical position of surgeon hand relative to tissue. By moving surgeon hand a linear array of craters is optionally produced, potentially resulting in a linear incision.

In some embodiments of the invention tissue contact sensing may be achieved by measuring an electrical impedance between a metallic surgical probe and tissue. As long as the probe tip is not in contact with tissue, electrical resistance may be approximately infinitely large. Upon contact, electrical impedance is reduced dramatically, depending upon tissue type. There are many cases where such a technique is inferior to tissue mechanical impedance sensing, such as sensing a conducting dental implant.

In some embodiments of the invention, a reduction of mechanical impedance while moving forward is measured. In such a case the change of impedance is an indication that the treatment tip has drilled a hole in a body membrane such as the tympanic membrane in case of myringotomy and reached a tissue cavity. Once such a measurement is detected, the motor controller may optionally commands backward retraction.

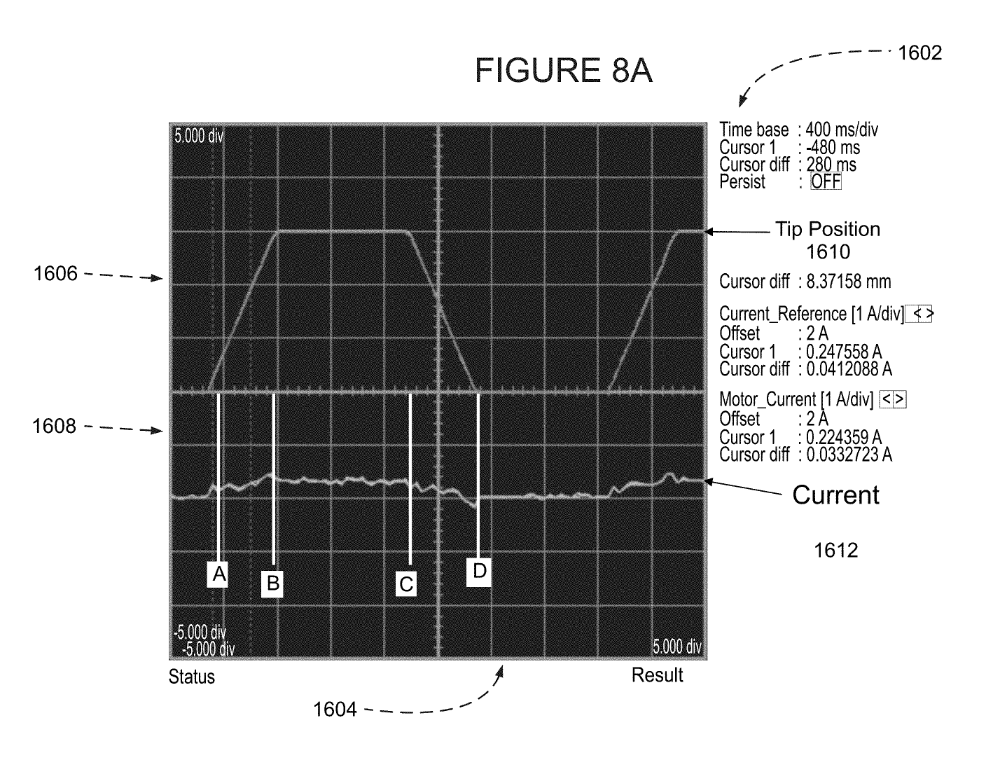

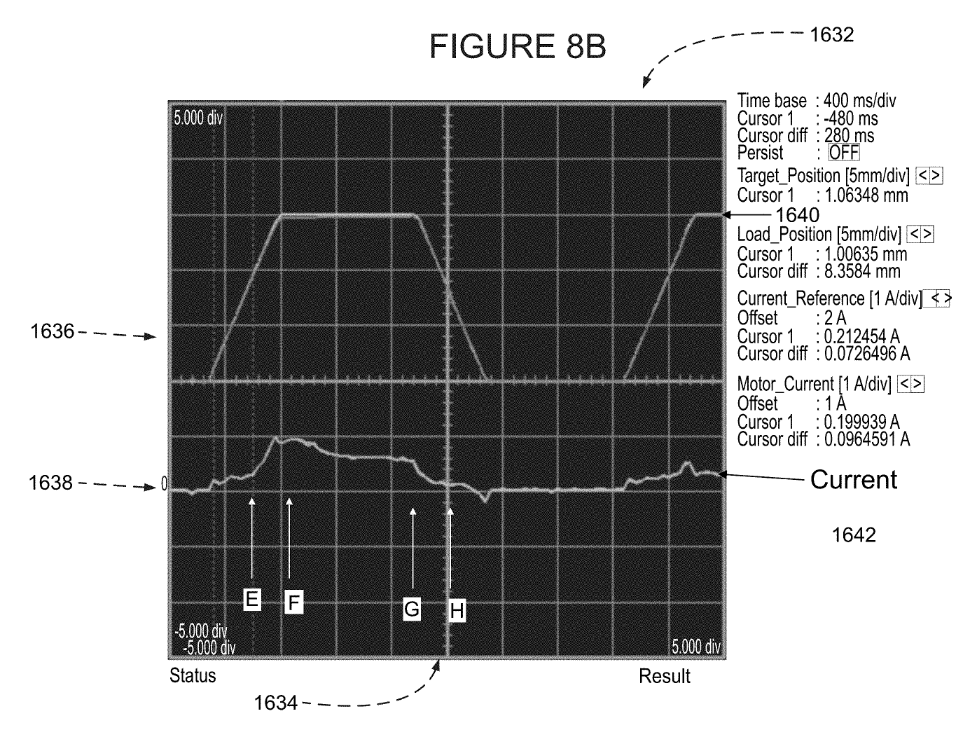

Reference is now made to FIG. 8A, which is an oscilloscope trace 1602 of a position of an array of tips and of a driving current of a linear motor driving the array of tips in air according to an example embodiment of the invention.

Reference is additionally made to FIG. 8B, which is an oscilloscope trace 1632 of a position of an array of tips and of a driving current of a linear motor driving the array of tips including a period of time touching impeding skin according to an example embodiment of the invention.

FIGS. 8A and 8B have X-axes 1604 1634 of time, 400 milliseconds per division and Y-axes of tip position 1606 1636, 5 mm per division, and driving current 1608 1638, 1 A per division, of a linear motor controlled using a closed loop control method of Pulse Width Modulation (PWM).

FIG. 8A depicts an upper trace 1610 showing tip position as a function of time, with the tips moving in air. Section AB of the upper trace 1610 corresponds to the tips advancing, section BC of the upper trace 1610 corresponds to the tip at maximal advance, and section CD of the upper trace 1610 corresponds to a retraction phase of the tips.

FIG. 8A depicts a lower trace 1612 showing a driving current used to advance the tips. The driving current depicted by the lower trace 1612 appears substantially constant, barring noise artifacts. The driving current depicted by the lower trace 1612 corresponds to mechanical impedance to the tip movement by tissue—no skin contact.

FIG. 8B depicts an upper trace 1640 showing tip position as a function of time, with the tips moving into contact with tissue, in the example of FIG. 8B the tissue is the skin of a finger placed in the path of the tips. Section EF of the upper trace 1640 corresponds to the tips advancing into the tissue, and section GH corresponds to tip retraction.

FIG. 8B depicts a lower trace 1642 showing a driving current used to advance the tips. The driving current depicted by the lower trace 1642 appears shows a current increase in the section EF. The advancing tip came into contact with the skin at point E and gradually pushed the skin while compressing it. In the example embodiment depicted by FIG. 8B the tip speed is controlled to be constant, as may be seen by the constant slope of the upper trace 1640 over the section EF. The driving current is proportional to the driving force, which is proportional to a resisting force in order to maintain the speed, and the resisting force is believed to be proportional to depth. The driving force and current reach a maximum at point F, which corresponds to the deepest depression. FIG. 8B shows a capability of detecting contact with skin as well as optionally determining a depth of depression based on force feedback which in some embodiments relates to the driving current.

In FIGS. 8A and 8B, the inventors have tested the capability of implementation of tissue contact detection as proposed above with a linear motor controlled by PWM of driving current. The upper trace 1640 of FIG. 8A describes tip position as function of time in air. Section AB is an advancing section, BC is maximal advance and CD is a retraction phase. The lower trace shows the driving current which is essentially constant. This indicates no mechanical impedance—no skin contact. FIG. 8B shows tip position (upper curve 1640) as in FIG. 8A in a case where tissue (finger skin) is located in front of the advancing tips. A lower trace 1642 shows the driving current.

As can be seen, there is a linear current increase in the section EF. The advancing tip got into contact with the tissue at point E and gradually pushed forward the tissue while compressing it. Since the driving current is proportional to the driving force (which is proportional to the resisting force which is proportional to depth), the driving force and current reaches a maximum at point F which is the deepest depression. We have thus confirmed the capability to detect skin contact as well as depth of depression with the force feedback which controls driving current.

Description of Example Embodiments of Treatment Tip(s) Construction

According to an embodiment of the invention a distal end of a treatment tip may have the following shapes: conical, pyramidal, round (spherical), cylindrical flat, chisel or blade. Tips may be hollow with a metallic envelop or non hollow solid. The treatment tips external surface may be biocompatible at working temperature of 300-850 deg C. during an entire procedure duration such as 10-30 minutes. According to one embodiment of the invention tip material should have high thermal conductivity in order to allow fast transfer of heat to vaporized crater volume and supply a latent heat of vaporization necessary to vaporize crater. One material suitable for tissue vaporization at high temperatures as well as fulfilling biocompatibility requirements is Tungsten, which thermal conductivity is ˜170 W/m sec. A flat tip made of Tungsten at 400 deg C. is capable of vaporizing ˜30-50 micron of tissue within ˜1 millisec. However, by using a Tungsten tip of conical shape or pyramidal shape, vaporization depth triples and can reach ˜150 micron. This is due to a geometric property of cones or pyramids to have a volume equal to ⅓ of the volume of a cylinder of identical height and diameters.

Production of low cost conical or pyramidal Tungsten tips of base diameter/width ˜300-1000 micron and height 1000 micron may be challenging. In some embodiments the Tungsten tip is machined. In some embodiments, a conical or pyramidal tip is optionally hollow as presented in FIGS. 1A and 2A, and is optionally produced by coining or stamping techniques or sintering techniques. In some embodiments, a distal end diameter of the conical or pyramidal tip is ˜100-300 micron.

Another highly thermally conductive tip material is copper (˜400 W/m deg C.). However since copper is not biocompatible, the pyramidal or conical non hollow tip may optionally be surrounded (coated) with a biocompatible material at high temperatures such as Titanium. Coating the above described tip dimensions made of copper with a titanium pyramidal envelope without significantly lowering thermal conductivity, may be done with an envelope thickness of approximately 50-150 micron. The titanium envelope may be produced by machining or by coining or stamping techniques. The copper core material of the tip may be produced by sintering techniques.

In some embodiments, the tip is a titanium hollow pyramid or cone of thickness 50-300 micron, preferably 50-100 micron.

In some embodiments, tip material is copper and a biocompatible coating is gold, optionally plated onto the copper. In one embodiment of the invention, a gold layer is not homogeneous: plating thickness is close to 100 micron on distal end of the tip which is in contact with the tissue, while thickness is only ˜5 micron at the tips base. A gradual change of gold plating thickness potentially ensures high stability of high temperature gold where contact with tissue is created, while material amounts and costs are potentially kept low due to a small thickness of plating close to the tip base (including a large part of tip surface area).

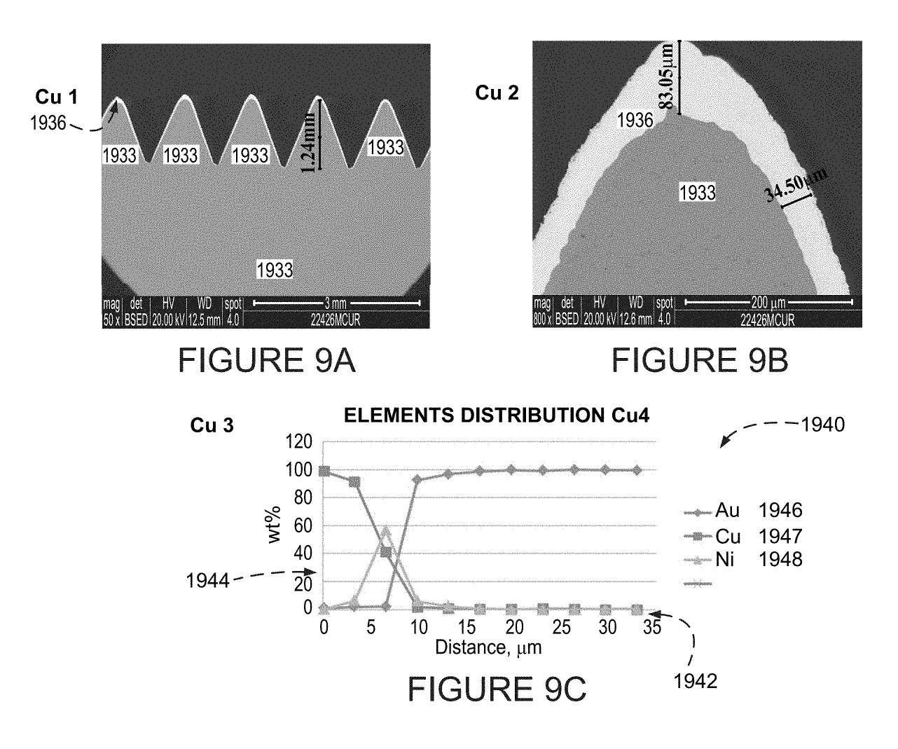

Reference is now made to FIGS. 9A and 9B, which are cross section images depicting copper tips 1933 coated with a coating 1936 of nickel followed by gold according to another example embodiment of the invention.

FIG. 9A depicts a copper base 1933 and copper tips coated with the nickel followed by gold coating 1936.

FIG. 9B depicts an enlarged section of FIG. 9A, showing one tip 1933, and the nickel followed by gold coating on the tip 1936. FIG. 9B shows an approximately 83 micron thick coating at the tip and an approximately 34 micron thick coating on the sides of the tip.

Reference is now made to FIG. 9C, which is a graph 1940 depicting concentration of elements as a function of distance along the copper tips and the nickel followed by gold coating of the example embodiment of FIGS. 9A and 9B.

The graph 1940 has an X-axis 1942 of distance in microns, and a Y-axis 1944 showing percentage of the elements in the material at the distance measured.

A first line 1946 in the graph 1940 shows concentration of Gold (Au).

A second line 1947 in the graph 1940 shows concentration of Copper (Cu).

A third line 1948 in the graph 1940 shows concentration of Nickel (Ni).

In the sample of the example embodiment of FIGS. 9A 9B and 9C the gold layer is 83 micron thick at the tip, and a layer of over 60 micron of pure gold is present although the tips were heated to a temperature of 520 degrees C. for 50 minutes. Since in some cases a duration of a skin rejuvenation treatment may last close to 20 minutes, the inventors heated the tips for a duration longer than 20 minutes.

In some embodiments, the array of tips is produced by using sintered copper tips which are electro-coated coated with a 6-20 micron nickel layer and further electro-coated by a 5-10 micron gold layer. It is noted that electroplating may produce a thicker coating at the tips, which are sharp and concentrate electric field. It is believed that electroplating the tips produces a synergy whereby the thicker plating is located where the array meets the tissue, and that the bio-compatible plating over a sintered array of tips is preferably formed by electroplating.

In order to test that the copper and nickel do not diffuse into the gold layer the array of tips was heated to a temperature of 520 degrees C. for a duration of 50 minutes and tested with an electron microscope for gold layer stability and with X-ray spectroscopy for Cu, Ni and Au concentrations as function of depth. The result showed high gold stability even at the sharp distal end of the tips as well as no diffusion of Cu or Ni to the surface.

A similar test was performed with a sintered array of stainless steel tips with good results.

FIGS. 9A and 9B present the cross section of a copper tip array plated with gold (each single tip in surgical incision unit may have same properties as each tip in an array of identical tips). The tip has been heated to a temperature of 500 degrees for a duration of 3 hours and tested for diffusion of nickel from nickel plating layer beneath the gold layer as well as copper ions. Diffusion didn't get close to external surface and tips were also tested for toxicity and biocompatibility. Results confirmed both stability and biocompatibility.

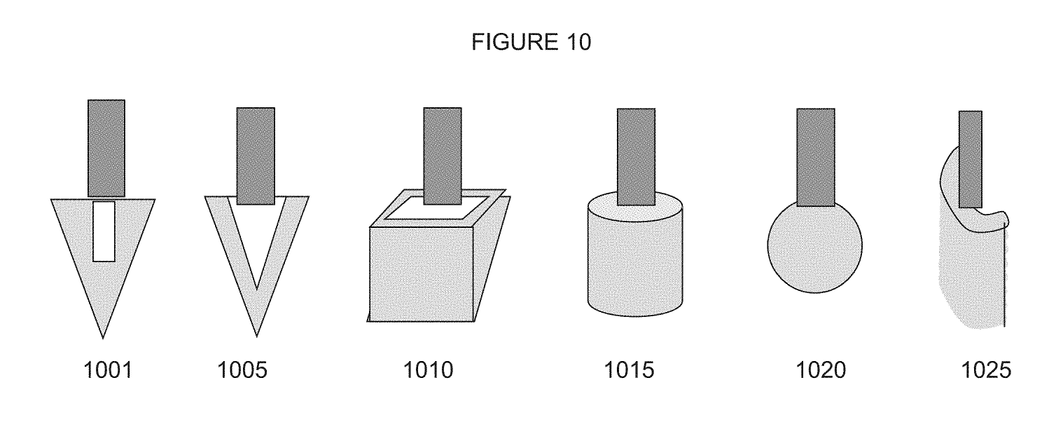

Reference is now made to FIG. 10, which is a simplified illustration of treatment probe tips according to example embodiments of the invention.

FIG. 10 presents various shapes of surgical tips 1001 1005 1010 1015 1020 1025 according to various embodiments of the invention. The shapes are: a-semi hollow conical 1001; b-hollow conical 1005; c-chisel 1010 (may be sharp for incision also without heat or not sharp enough for cold incision) d-cylindrical 1015 (solid or hollow); e-spherical 1020 (potentially for use in opening ducts such as blood vessels) f-banana shape 1025—potentially used for myringotomy. In some embodiments the tips are metallic and opaque. In some embodiments the tips are made from sapphire and/or diamond and/or ceramics such as ALN (Aluminum Nitride).

Crater vaporization experiments with various tips materials have been performed by the inventors in order to confirm the dimensions and material selections of tips as described above. Experiments were performed on human skin.

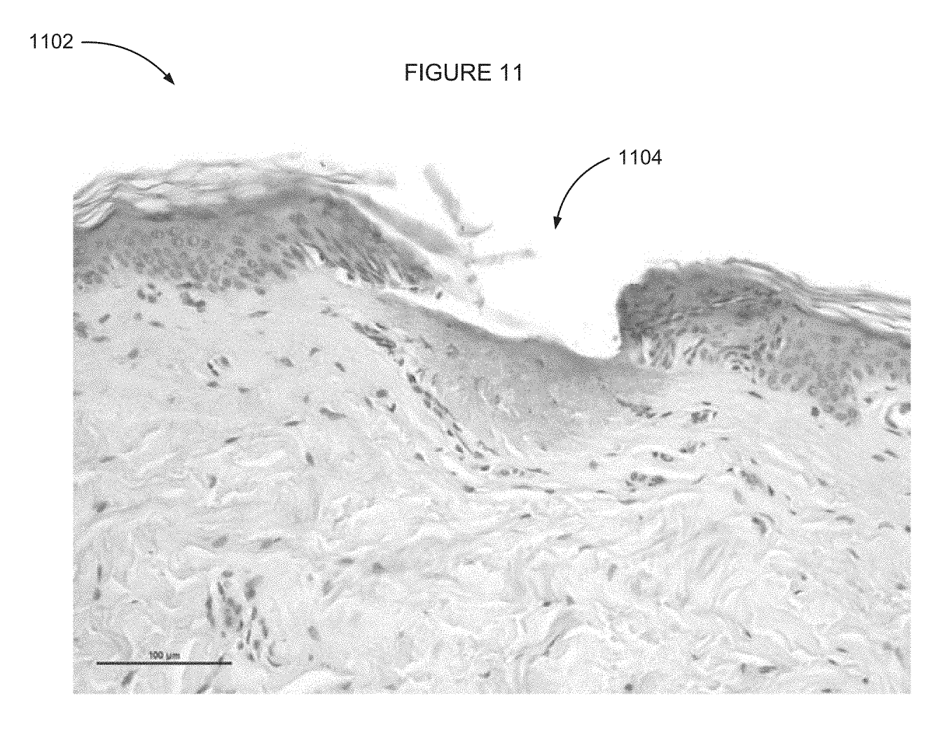

Reference is now made to FIG. 11, which is a photograph of a histology cross section of an in vivo human skin vaporized crater produced immediately after treatment according to an example embodiment of the invention.

FIG. 11 presents a histology cross section 1102 of in vivo human skin vaporized crater 1104 immediately after treatment. The crater 1104 depth is ˜100 micron. The crater 1104 diameter is ˜150 micron. The crater 1104 shape is conical. Collateral damage is mostly less than 80 micron except at the crater 1104 center where it is ˜100 micron. There is no peripheral carbonization. The crater 1104 has been obtained with a copper tip plated with gold and operated at 400 deg C.

In another embodiment of the invention treatment tip can be cleaned and sterilized by heating the tip above 450-500 deg C. for a duration of 2-5 minutes. At that temperature carbon present in organic material combust and organic material is totally ablated. Inventors have tested that sterilization technique with success.

Some Example Applications:

Dentistry and Oral Maxillofacial Surgery:

Reference is now made to FIG. 12, which is a simplified cross-sectional illustration of an example application of incising tissue according to an example embodiment of the invention.

FIG. 12 presents an example of a dental application whereby tissue 122 has to be delicately incised over a titanium metallic implant 121 in order to insert an artificial tooth (following healing from first surgical implantation step). A tip 60, which is optionally chisel-shaped, incises an incision 61 above the dental implant 121. A pulling of tissue 122 with a forceps optionally ensures clear tissue exposure and a capability to incise the tissue 122 layer by layer. Shallow thermal damage further enables to accurately incise tissue layer after layer, each optionally of depth D, until the metallic implant 121 is revealed and exposed. There is potentially no bleeding, and healing from the incision 61 is fast due to very shallow thermal damage. Monopolar devices can't be used in such cases, and sapphire tips or bipolar ESU units produce more thermal damage followed by slower healing. The quantity of heat transferred to the implant 121, in the above example embodiment, is small enough to avoid damage due to contacting the implant 121 surface, due to small contact duration, potentially thanks to optional mechanical impedance sensing, and to a limited volume of the treatment tip 60 which is smaller than the implant 121 volume.

In some embodiments a vaporizing element optionally includes a linear array of tips such as 2 to 10 tips. The linear array potentially enhances incision speed. An example of an incision of a calf liver with an array of 4 tips is described below. In the example described below the incision depth was approximately 1 mm, the incision length was 4 cm, the incision duration was 2 seconds, thermal damage was less than 100 microns, and tip temperature was 400 degrees Celsius.

In some embodiments a bottom of a hollow channel in a tooth is optionally heated. Such an embodiment is optionally used for sterilization of the bottom of the channel. Sterilization of the channel bottom with current cold devices is difficult. Using laser light emitted from a thin fiber placed near the bottom of the channel may slowly heat the bottom while creating collateral heating and damage. By introducing a coated light-guide/fiber according to an embodiment of the invention into the channel down to its bottom and activating the distal tip the channel bottom is optionally contacted for a short and measurable duration, optionally measured by resistance to tip movement, and sterilize the channel bottom at a temperature between ˜300-800 deg C. without damaging tissue.

Laparoscopic and Endoscopic Surgery:

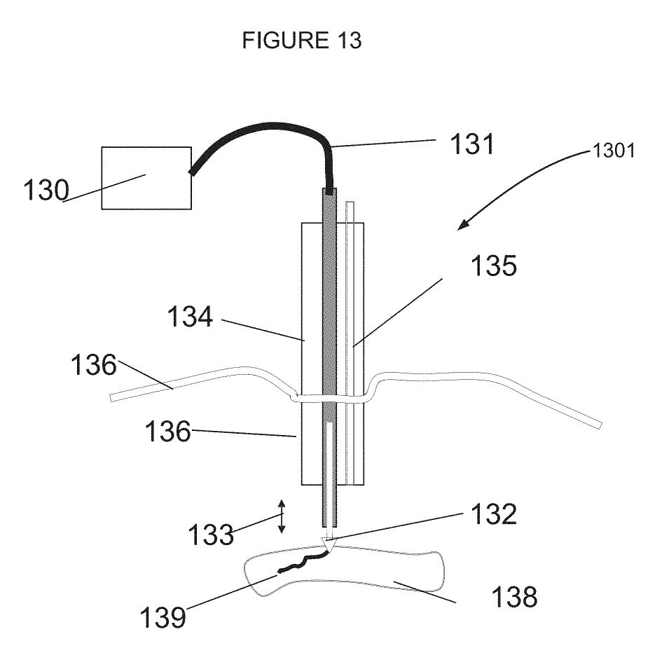

Reference is now made to FIG. 13, which is a simplified cross-sectional illustration of an example application of incising tissue according to an example embodiment of the invention.

FIG. 13 presents an application of some embodiments of the invention, in a mode suitable for, by way of a non-limiting example, laparoscopic and endoscopic surgery. As a non-limiting example, incision of a wall of a fallopian tube in laparoscopy is described. A surgical unit 1301 includes a laser or electrical heater unit 130, which optionally also incorporates a master clock and a motor controller. The motor may be located either in the unit 130 or closer to a distal tip 132. An energy delivery fiber or cord 131 leads to a laparoscope 134. The distal tip 132 is optionally heated by a train of energy pulses and optionally synchronously oscillates 133. The laparoscope 134 may also include an optical viewing channel 135. The laparoscope is optionally inserted into a body through a puncture in abdominal skin and sub layers 136. The treated organ may be a fallopian tube 138 which is delicately incised 139.

In some embodiments additional endoscopic applications include an incision of gallbladder, incision of adhesions, incision of polyps in intestines, incision or vaporization of tumors in a trachea, among many others.

Drug Introduction through Human Tissue:

In some embodiments an array of tips is used for introduction of drugs through tissue, in some embodiments even with deep crater vaporization. As an example, inventors have vaporized arrays of 9×9 craters through the epidermis of a male arm. The craters were measured to have open diameters of 200 to 300 microns. The craters were observed to remain open for a duration of 6 hours. The diameter of each crater was measured as function of depth in the skin with a confocal microscope (mavrick). After 6 hours a drug, in this case liquid yellow florescene—Floreszein SE Thilo Germany—was applied to the skin. The drug was fully absorbed within 2 minutes.

It is noted that a duration during which craters remain open is of significance in drug delivery. The duration potentially depends on opening a crater without incurring deep collateral damage, which might serve as a barrier to drug transfer, and on the crater diameter. By way of a non-limiting example, shallow craters, craters in the stratum cornea only, with too small a diameter may close within a too short duration, such as 30 minutes, before drug is applied.

Description of Additional Example Embodiments

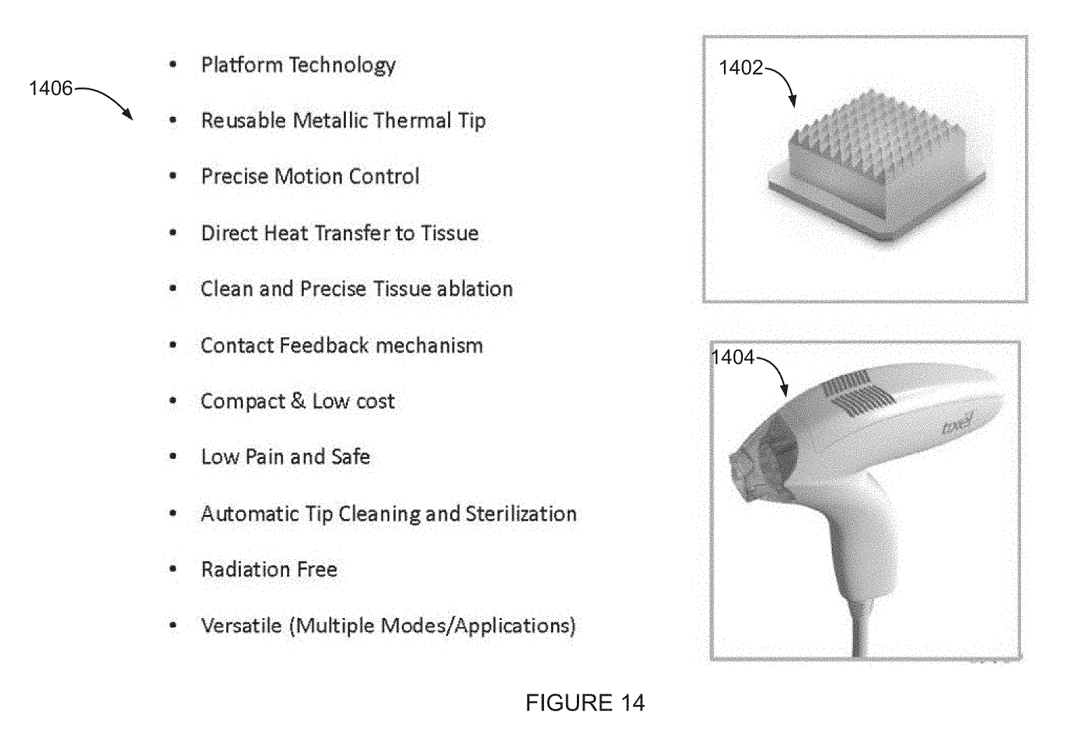

Reference is now made to FIG. 14, which is a simplified illustration of an array of tips 1402, a hand-held device 1404, and a list of features 1406 of the hand-held device 1404, according to an example embodiment of the invention.

The list of features includes:

Platform technology, the same unit may optionally be used in different applications, for example in different surgical applications, by changing a length and/or a shape of a metallic sheath and/or selection of a heated tip type and/or on operating parameters. The same platform may optionally be used in aesthetics as well as in drug delivery.

Reusable. By way of a non-limiting example a metallic thermal tip—in some embodiments the treatment tips are reusable. The treatment tips may optionally be sterilized between uses. The treatment tips are optionally sterilized and/or cleaned of any residue by heating to high temperatures which evaporate residue and sterilize the tips, optionally using the device 1404 itself;

Precise motion control—as described above, depth of penetration of the treatment tips may optionally be precisely controlled;

Direct heat transfer to tissue;

Clean and precise tissue ablation;

Contact feedback mechanism;

Compact and low cost;

Low pain and safe;

Automatic tip cleaning and sterilization;

Radiation free; and

Versatile (multiple modes and applications).

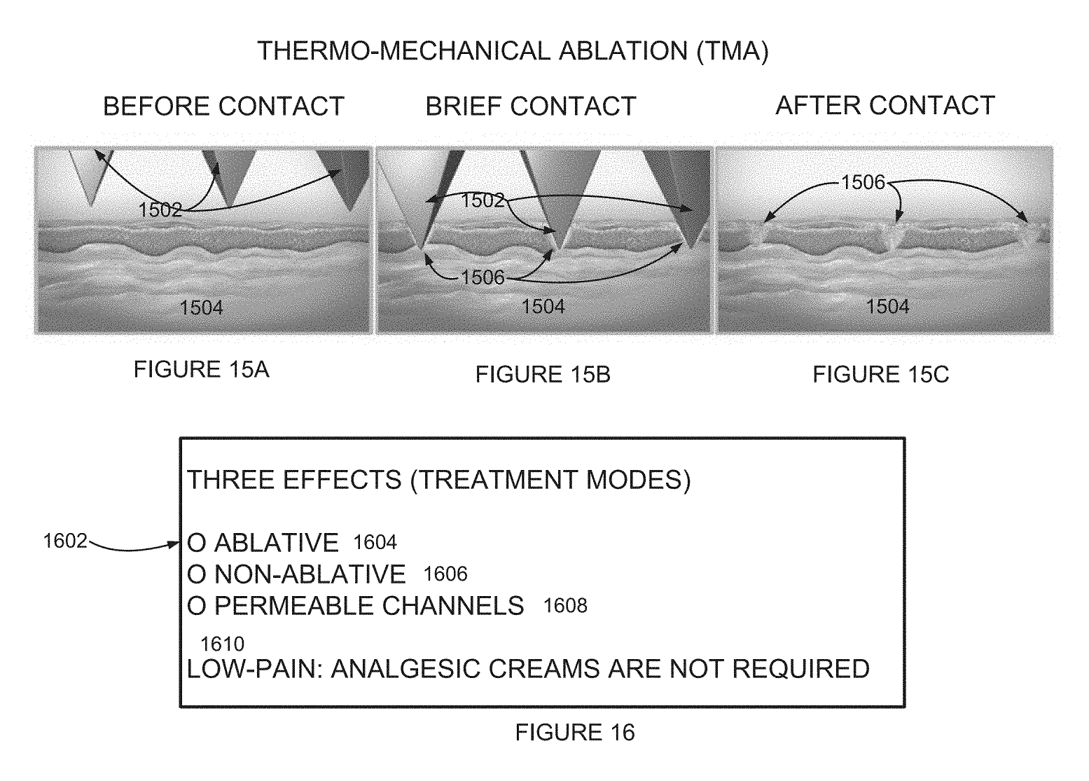

Reference is now made to FIGS. 15A, 15B and 15C, which are simplified illustrations of a process of thermo-mechanical ablation (TMA) according to an example embodiment of the invention.

FIG. 15A depicts a simplified illustration of a tip array 1502 before contact with tissue 1504.

FIG. 15B depicts a simplified illustration of the tip array 1502 of FIG. 15A during a brief contact with the tissue 1504. During at least some of the duration of the contact the tip array is optionally heated, thereby heating the tissue 1504 and producing craters 1506 in the tissue 1504.

FIG. 15C depicts a simplified illustration of the tissue 1504 with craters 1506 which were formed by the tip array 1502 while the tip array 1502 was heated and in contact with the tissue 1504.

Reference is now made to FIG. 16, which is a simplified illustration of a list 1602 of various effects produced by applying heated tips according to an example embodiment of the invention.

The list 1602 enumerates three effects, or treatment modes, including:

an ablative and/or vaporizing effect 1604 on tissue. A vaporization depth may vary, by way of a non-limiting example, from 20 microns to 500 microns, potentially depending on operating parameters and/or on a number of treatment pulses at the same spot;

a non-ablative effect 1606 on tissue whereby the tissue's outer layer is not vaporized while underlying holes may optionally be produced. Such an effect may happen for example while treating skin, where the stratum cornea, which is more difficult to vaporize, is not vaporized, while the epidermis, which has a higher water content, does vaporize. In some embodiments the above-described effect produces skin treatment and self-bandage, that is, the stratum corneum acts as a cover to the treated epidermis. In some embodiments the above-described effect is optionally achieved during a shorter duration than with the ablative effect; and

production of permeable channels 1608 in tissue, which permeable channels may serve to introduce drugs into the tissue.

A benefit 1610 of the treatment modes is also listed, namely that the treatment modes produce little or no pain, and therefore potentially do not require use of an analgesic.

In some embodiments permeable skin or tissue is produced by vaporization of the stratum corneum and optionally some epidermis without coagulation and without producing damage below the epidermis.

In some embodiments the above is optionally achieved by a short duration of contact between the tissue heating element and the tissue, such as, by way of a non-limiting example, below 10 milliseconds, or below a range of 1-200 milliseconds.

In some embodiments the above is optionally achieved by using a sharp distal end of a tip of the tissue heating element, such, by way of a non-limiting example, a width of a distal end of a tip of the tissue heating element is less than 150 microns, or less than 100 microns, or less than 50 or 20 microns.

In some embodiments the above is optionally achieved by using a tip with relatively low heat conduction, at least as compared to copper. By way of a non-limiting example, the tip may be composed of stainless steel and/or titanium.

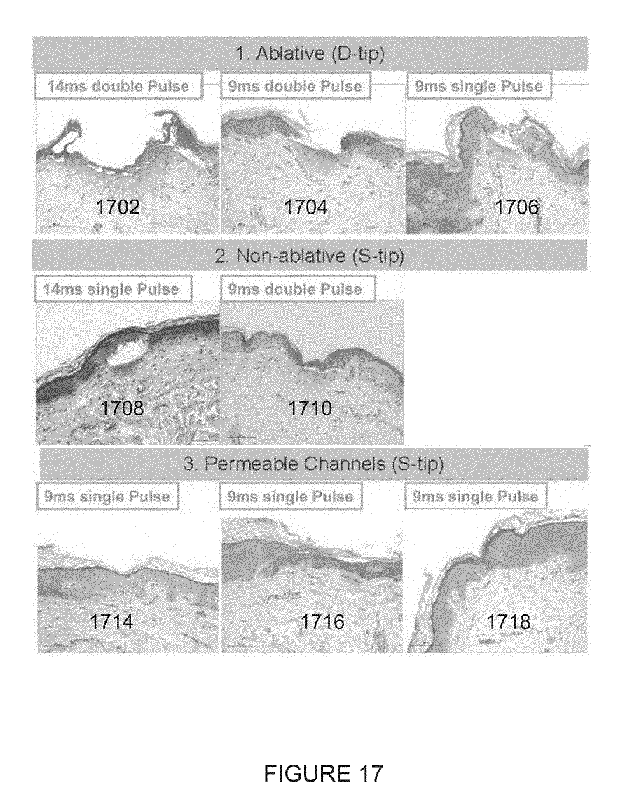

Reference is now made to FIG. 17 which includes nine cross section images depicting tissue treated in various treatment modes using various treatment tips according to various example embodiments of the invention.

FIG. 17 depicts:

A first cross-sectional image of tissue 1702 which has been treated with an ablative D-type tip, using an example embodiment of a copper tip coated with gold, with two heating pulses of 14 milliseconds each and a crater formed in the tissue 1702;

A second cross-sectional image of tissue 1704 which has been treated with an ablative D-type tip with two heating pulses of 9 milliseconds each and a crater formed in the tissue 1704;

A third cross-sectional image of tissue 1706 which has been treated with an ablative D-type tip with a single heating pulse of 9 milliseconds and a crater formed in the tissue 1706;

A fourth cross-sectional image of tissue 1708 which has been treated with a non-ablative S-type tip, using an example embodiment of a stainless steel tip coated with gold, with a single heating pulse of 14 milliseconds and a crater formed in the tissue 1708;

A fifth cross-sectional image of tissue 1710 which has been treated with an S-type tip with two heating pulses of 9 milliseconds each and a crater formed in the tissue 1710;

A sixth cross-sectional image of tissue 1714 which has been treated with an S-type tip for producing permeable channels with a single heating pulse of 9 milliseconds and a crater formed in the tissue 1714;

A seventh cross-sectional image of tissue 1716 which has been treated with an S-type tip for producing permeable channels with a single heating pulse of 9 milliseconds and a crater formed in the tissue 1716; and

An eighth cross-sectional image of tissue 1718 which has been treated with an S-type tip for producing permeable channels with a single heating pulse of 9 milliseconds and a crater formed in the tissue 1718.

In the present application and claims probe tips are also referred to using the following terms:

a D-type tip having relatively high thermal conductivity, above ˜150 W/deg C. m;

an S-type tip having relatively lower thermal conductivity such as ˜20-150 W/mDeg C.; and

a T-type tip, made of Titanium, and generally similar to an S-type tip, having relatively lower thermal conductivity such as ˜20-150 W/mDeg C.

The D-type tip is potentially more suitable for an ablative, vaporizing, treatment mode. The S-type tip is potentially more suitable for a non-ablative treatment mode.

Some comments are hereby made about use of S-type non ablative tips:

in some embodiments, potentially more so when using a double pulse, the stratum cornea is potentially wholly or partially removed, potentially resulting in an ablative effect without the stratum corneum remaining as a potential bandage for a produced crater;

in some embodiments, potentially more so when using a single pulse, such as the single pulse of 9 millisecond duration used for producing images 1714 1716 1718 with an S-type tip, drug permeation is thought to be potentially enabled by reorganization of the cells in both the stratum corneum and the epidermis, potentially without coagulation which might serves as a barrier to drugs. The reorganization potentially allows drug permeation through the treated skin.

In some embodiments, such as when S-type tips are sharp, for example with ˜100 micron distal diameter, the sharp tips may ablate the stratum corneum when 9 millisecond pulse duration is used. However, although an ablative effect occurs, a produced crater appears to lack coagulative collateral damage boundaries. As a result, the produced crater is potentially permeable for a relatively long duration, up to 3, or 6, or 12 hours.

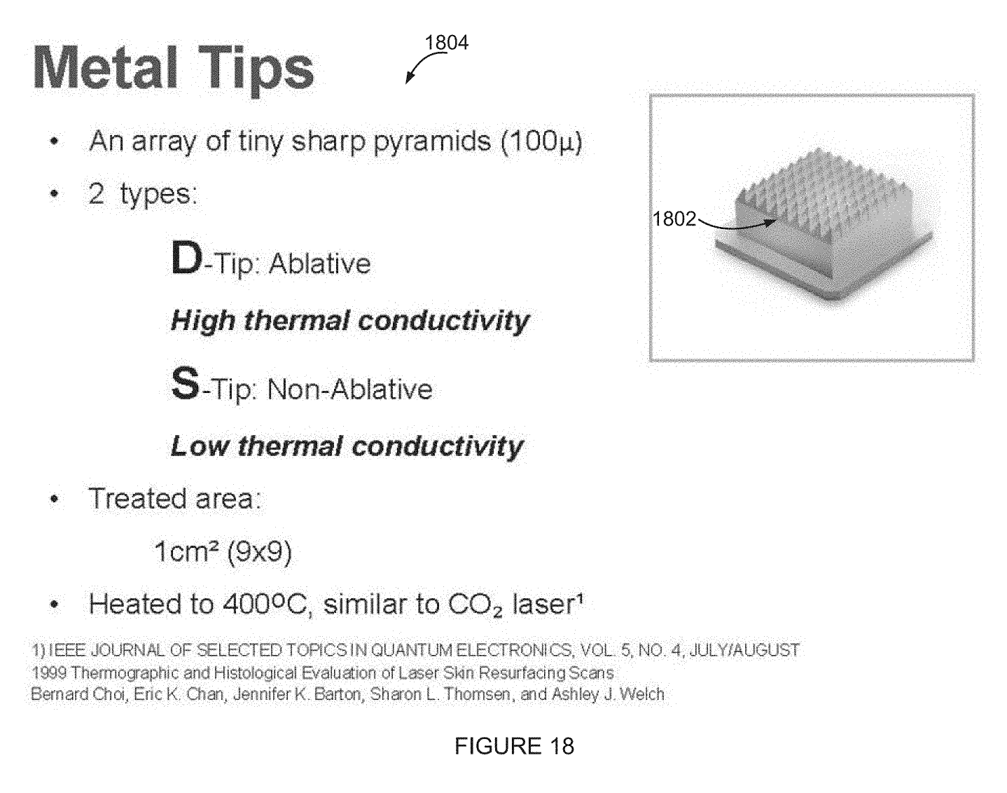

Reference is now made to FIG. 18, which is a simplified illustration of an array of tips 1802 and a description 1804 of the array of tips 1802 according to an example embodiment of the invention.

The description 1804 of the array of tips 1802 includes:

the array of tips 1802 is an array of metal tips;

the array of tips 1802 is an array of tiny sharp pyramidal tips having a width of approximately 100 micron, and a radius of curvature of approximately 50 microns. In various embodiments tip width can range between ˜100 and 1000 microns, and tip radius of curvature can range between ˜50 microns and flat surface (corresponding to an infinite radius of curvature).

It is noted that in some embodiments, a treated area produced by the array of tips 1802 which includes 9×9 tips as described above is approximately 1 square centimeter;

in some embodiments, the array of tips 1802 may be heated to a temperature of 400 degrees Celsius, which is similar to a temperature reached when treating tissue with a CO₂ laser.

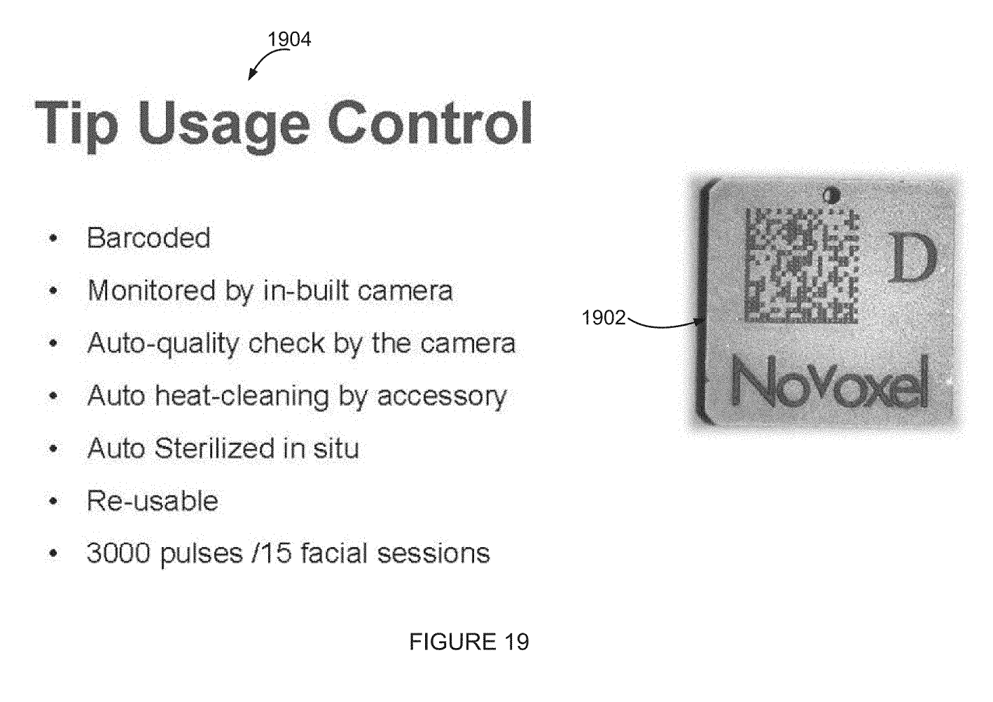

Reference is now made to FIG. 19, which is a simplified illustration of a barcode 1902 optionally used with an array of tips and a description 1904 of further optional features associated with the array of tips according to an example embodiment of the invention.

The description 1904 includes optional features associated with the array of tips according to an example embodiment of the invention:

the array of tips may optionally include a barcode for optionally describing dimensions, tip shape, individual tip identification number, and so on;

the array of tips and/or the barcode may be monitored by a built-in camera in a thermal surgical vaporization and incision of tissue system according to an example embodiment of the invention. The monitoring may be used to read the barcode and enter parameters into the system and/or to display the array of tips as it nears and contacts tissue;

in some embodiments the bar code is used for identification of a tip, which optionally serves to count a number of uses of the tip, and optionally used for limiting the number of uses of the tip to be less than a specified number of times. A limitation on the number of uses of a tip can potentially serve for preventing a potential long term damage to a coating of the tip after a specific number of uses at high temperature, such as at 400 deg C. A limitation on the number of uses of a tip may be, by way of a non-limiting example, 15 uses, or a range of 3-50 uses;

in some embodiments the bar code is used for recording which tip is used for which subject or patient. By way of a non-limiting example, a tip may be used for several treatments with the same patient, but may in some cases not be used with other patients;

in some embodiments the above-mentioned camera may perform an automatic quality check of the array of tips, such as, by way of some non-limiting examples, cleanliness of tips following a use, check if tip sharpness is preserved; if there is carbonization on the tip; if some tips within the array are possibly bent;

the array of tips may optionally be automatically cleaned by heating the array of tips to a temperature high enough to vaporize residue which may be left on the array of tips, and also sterilize the array of tips;

the array of tips is optionally re-usable;

the array of tips is good for at least 3,000 heat pulse and/or 15 facial treatment sessions.

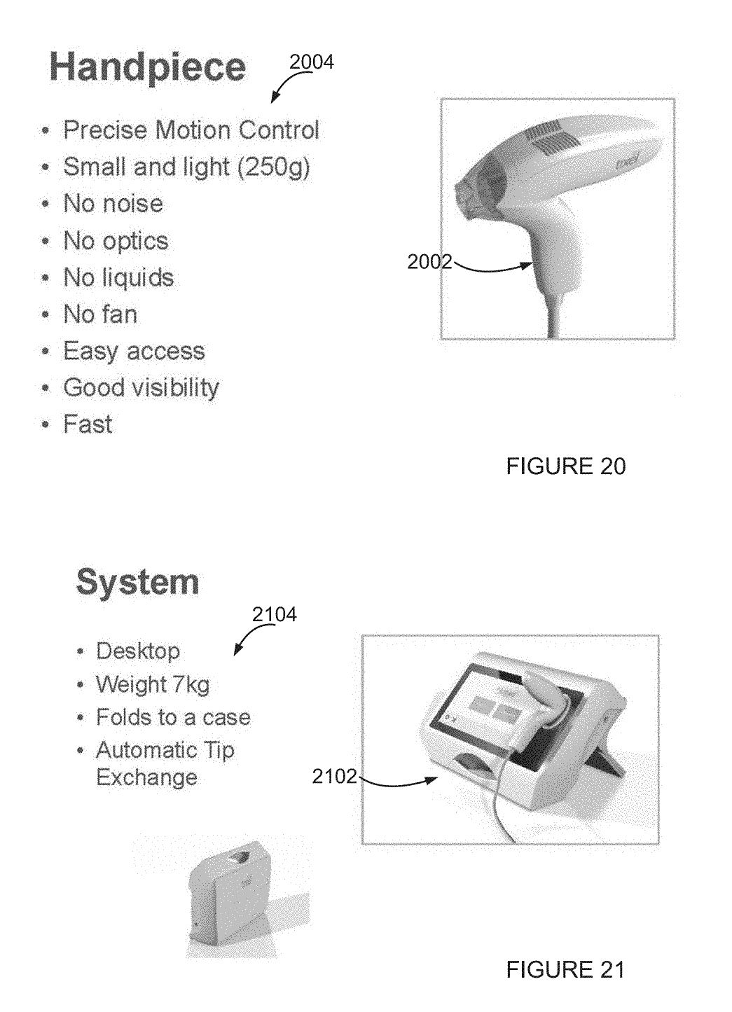

Reference is now made to FIG. 20, which is a simplified illustration of a hand-piece 2002 a description 2004 of optional features associated with the hand-piece 2002 according to an example embodiment of the invention.

The description 2004 includes optional features associated with the hand-piece 2002:

precise motion control of surgery by the hand-piece 2002 based on optional automatic depth control of incision, which, potentially by detecting contact with tissue and measuring additional advance beyond the point of contact, potentially attains a controlled depth regardless of tissue impedance;

the hand-piece 2002 is small and light (for example 250 g) relative to current instruments for thermal surgical vaporization and incision of tissue;

use of the hand-piece 2002 makes no noise;

in some embodiment use of the hand-piece 2002 requires no optics;

in some embodiment use of the hand-piece 2002 requires no liquids;

in some embodiment use of the hand-piece 2002 requires no fan;

the hand-piece 2002 shape, size, weight provides easy access to many surgical locations;

the hand-piece 2002 shape, size, weight provides good visibility of a treatment location; and

the hand-piece 2002 enables fast treatment. By way of a non-limiting example, a surgical incision of approximately 1 cm length may be performed in approximately 2 seconds, with a 100 micron depth. In such an example the repetition rate of the vaporization tip is 1 Hz, similar to a case of surface ablation in skin fractional vaporization. Small lesions of few mm size, such as, by way of a non-limiting example, 0.5-5 mm, height are optionally vaporized within less than a minute.

Reference is now made to FIG. 21, which is a simplified illustration of a system 2102 for thermal surgical vaporization and incision of tissue and a description 2104 of optional features associated with the system 2102 according to an example embodiment of the invention.

The description 2104 includes optional features associated with the system 2102:

the system 2102 may be compact enough to be deployed as a desktop system;

in some embodiments the system weight may be approximately 1-7 kg;

the system 2102 may optionally fold to a portable case for portability;

the system 2102 optionally provides capability for automatic tip exchange.

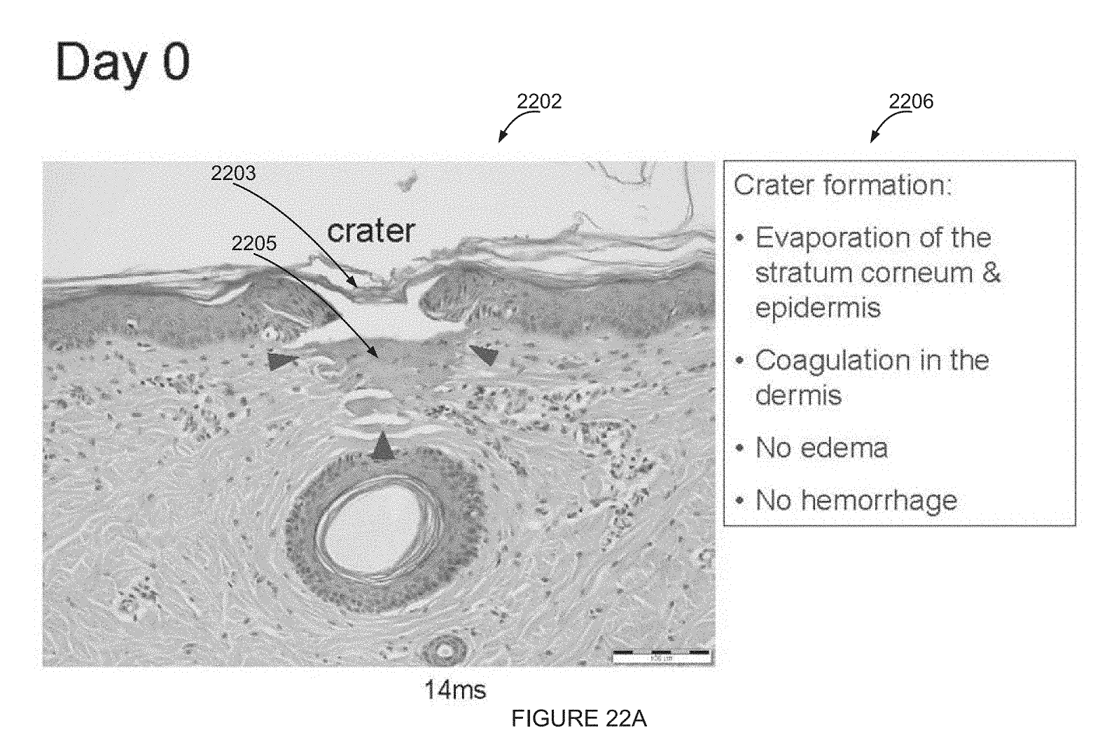

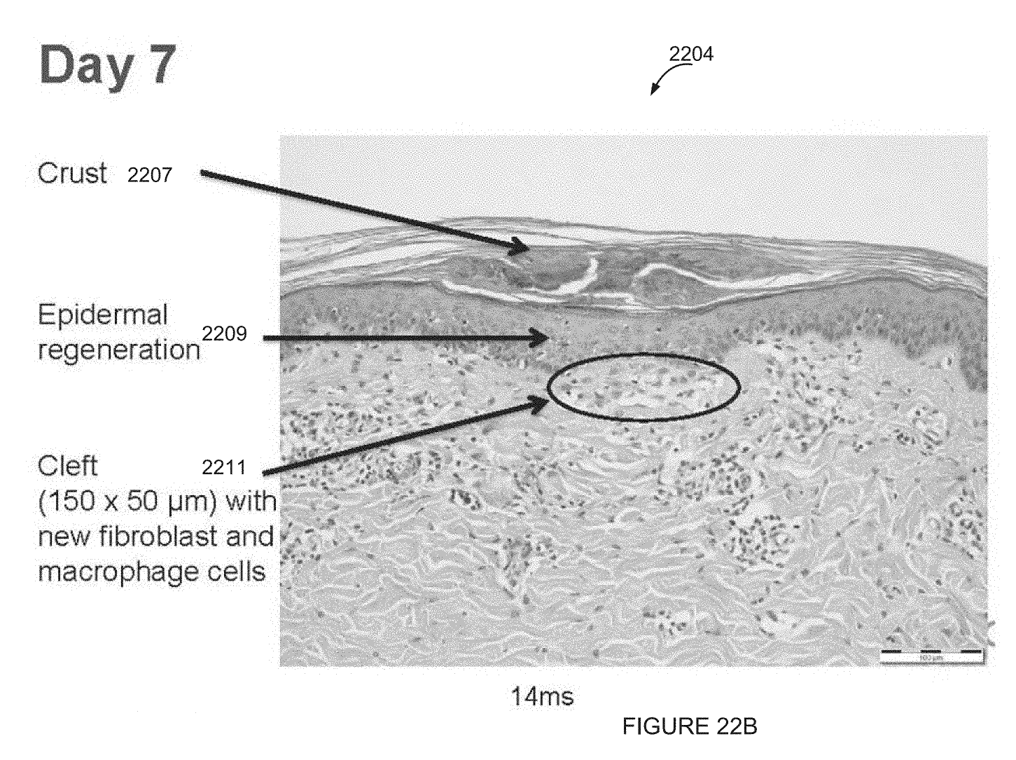

Reference is now made to FIGS. 22A and 22B which include cross section images 2202 2204 depicting tissue treated in an experiment and a description 2206 of findings associated with the experiment according to an example embodiment of the invention.

FIG. 22A depicts the image 2202 of a crater 2203 as produced on “Day 0”, the same day of treatment, caused by evaporation of the stratum corneum and the epidermis of skin tissue and a coagulation portion 2205 in the tissue. The description 2206 of the findings also reports that no edema and no hemorrhage were detected on Day 0. The treatment was a single 14 millisecond pulse of heat.

FIG. 22B depicts the image 2204 of the tissue depicted in FIG. 22A, on “Day 7”, seven days after treatment. The image 2204 shows a crust 2207 has developed, shows epidermal regeneration 2209, and shows a cleft 2211 having dimensions of 150 microns×50 microns with new fibroblast and macrophage cells.

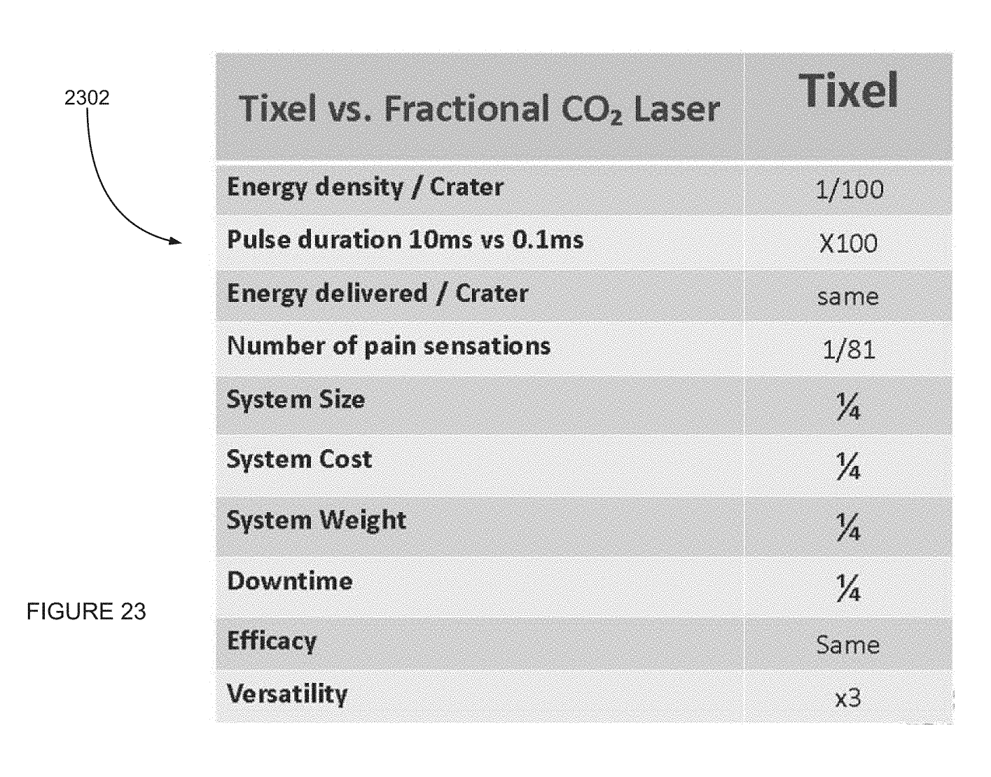

Reference is now made to FIG. 23, which is a table 2302 comparing treatment according to an example embodiment of the invention, named Tixel, to treatment with a Fractional CO₂ laser. The table 2302 compares treatments, skin cratering or vaporizing craters in skin, in the above-mentioned modes for delivering a same amount of energy per tissue crater produced:

energy density per crater in a Tixel treatment compared to Fractional CO₂ laser treatment is 1:100;

pulse duration in an example Tixel treatment is 10 milliseconds, compared to Fractional CO₂ laser pulse duration of 0.1 milliseconds, for a ratio of 100:1;

energy delivered per crater in a Tixel treatment compared to Fractional CO₂ laser treatment is the same;

a ratio of a number of pain sensations triggered in a Tixel treatment compared to a Fractional CO₂ laser treatment is, by way of a non-limiting example, 1:81, since the Tixel treatment can optionally produce an array of, for example, 81 craters within one potential pain event lasting a few milliseconds, while a CO₂ laser produces the craters sequentially, causing 81 potential pain triggers each lasting a few milliseconds;

a size ratio of systems for providing the above treatments in a Tixel treatment system compared to a commercial Fractional CO₂ laser treatment is 1:4;

a cost ratio of systems for providing the above treatments in a Tixel treatment system compared to a commercial Fractional CO₂ laser treatment is 1:4;

a system weight ratio of systems for providing the above treatments in a Tixel treatment system compared to a commercial Fractional CO₂ laser treatment is 1:4;

an expected downtime, typically patient stay-at-home time, ratio of systems for providing the above treatments in a Tixel treatment system compared to a commercial Fractional CO₂ laser treatment is 1:4;

an expected efficacy, or end result of treatment in a Tixel treatment system compared to a commercial Fractional CO₂ laser treatment is about the same;

an estimated versatility of a Tixel treatment system compared to a commercial Fractional CO₂ laser treatment is about threefold, based on smaller size and weight, lending itself to more uses and easier use.

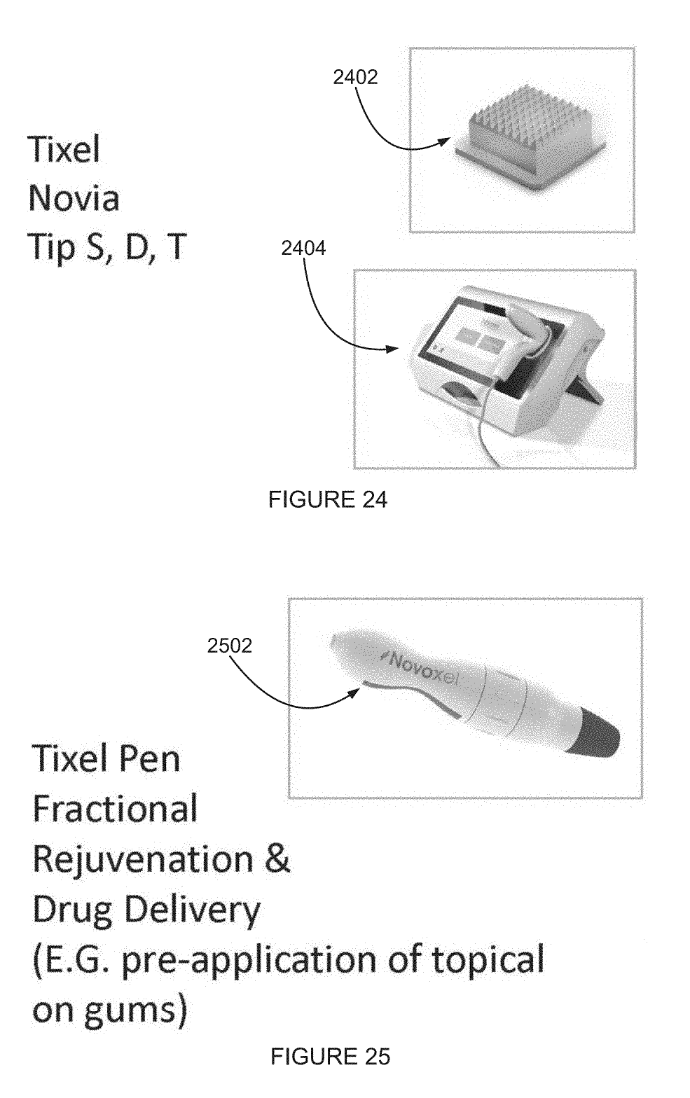

Reference is now made to FIG. 24, which is a simplified illustration of an array of tips 2402 and a treatment system 2404 according to an example embodiment of the invention.

FIG. 24 also includes text listing three non-limiting examples of three types of tips which may be sued in conjunction with the treatment system 2404, being an S-type tip (not shown); a D-type tip (not shown); and a T-type tip (not shown).

Reference is now made to FIG. 25, which is a simplified illustration of a hand-piece 2502 according to an example embodiment of the invention.

The hand-piece 2502 is a example “pen-shaped”, or elongated shape hand-piece, which is potentially suitable for working in some tighter, more constrained spaces, such as for production of craters on gums in a mouth, for example for application of a drug through the craters produced, or for producing an incision in gums.

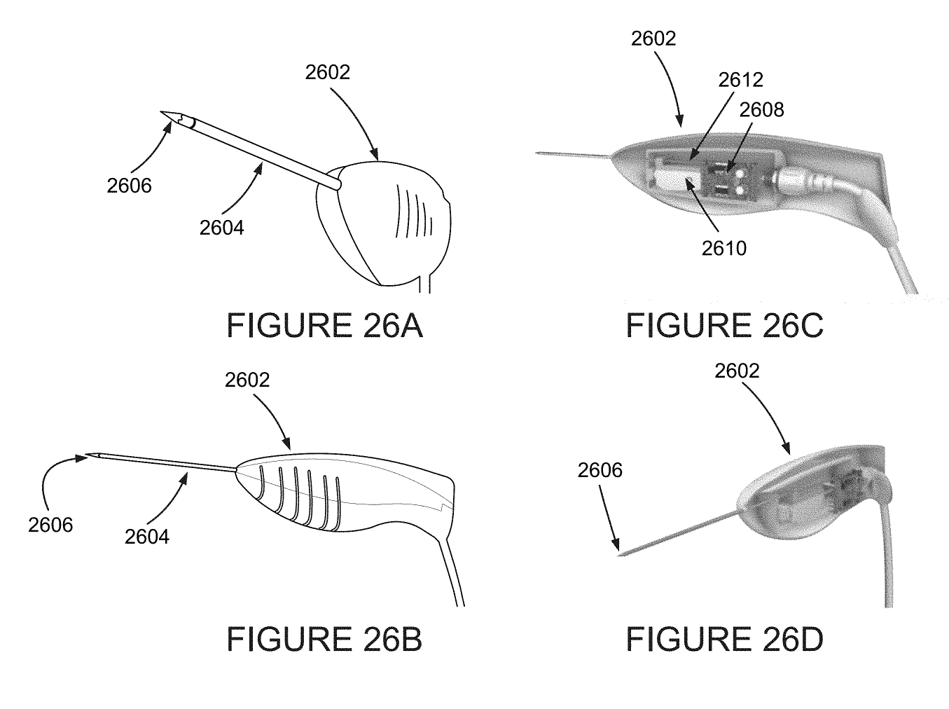

Reference is now made to FIGS. 26A-26D, which are simplified illustrations of a hand-piece 2602 according to another example embodiment of the invention.

The hand-piece 2602 includes an elongated thin and narrow extension, hereby termed sheath 2604, with a treatment tip 2606 at a distal end of the sheath 2604.

FIGS. 26A and 26B show a semi-frontal view and a side view of the hand-piece 2602.

FIG. 26C shows a side view of a cross section of the hand-piece 2602, including an electronic controller 2608, a mechanical oscillator 2610 or vibration driver 2610, and a laser delivery fiber 2612.

FIG. 26D shows a semi-frontal view of the cross section of the hand-piece 2602 which was shown in FIG. 26C.

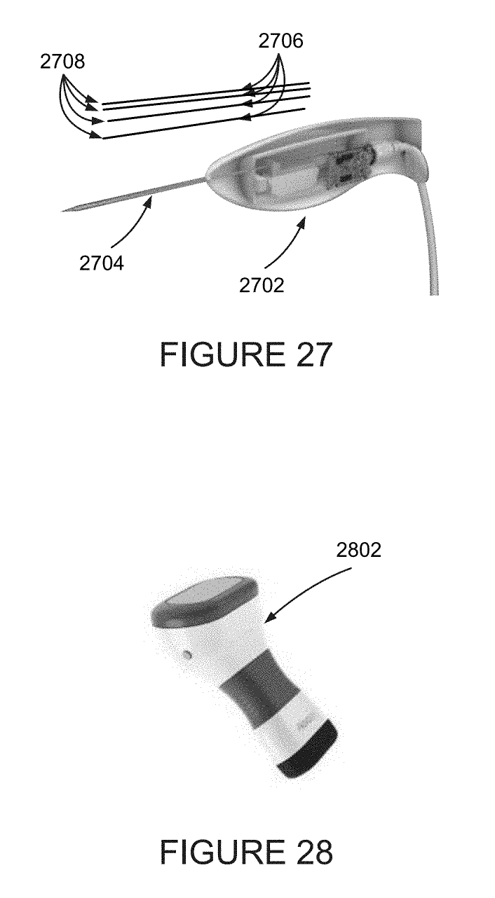

Reference is now made to FIG. 27, which is a simplified illustration of a semi-frontal view of a cross section of a hand-piece 2702 according to an example embodiment of the invention.

The hand-piece 2702 includes a replaceable elongated thin and narrow extension, hereby termed sheath 2704, and various additional extensions 2706 with different shaped tips 2708.

Reference is now made to FIG. 28, which is a simplified illustration of a hand-piece 2802 according to yet another example embodiment of the invention.

The hand-piece 2802 is optionally shaped for self administering skin cratering, potentially for home use. In some embodiments the hand-piece 2802 is shaped and configured with an array of tips for cratering skin, potentially for treating skin to improve drug passing through cratered skin.

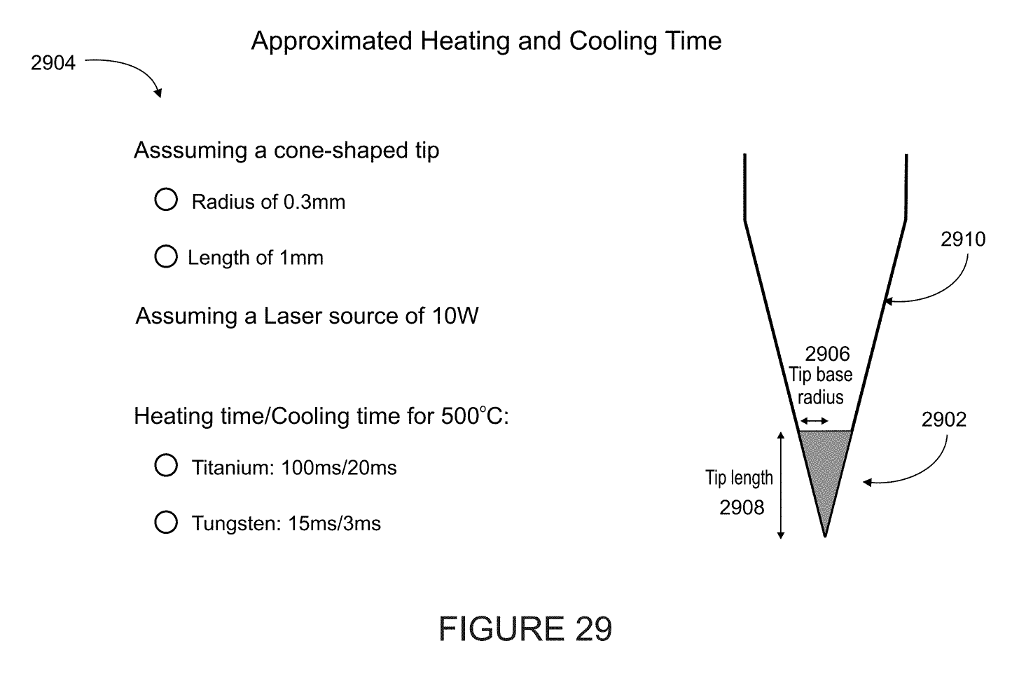

Reference is now made to FIG. 29, which is a simplified illustration of a tip 2902 and a tip base 2910 and a description 2904 of the tip 2902 according to an example embodiment of the invention.

The tip 2902 may be described by dimensions of a radius of curvature 2906 of the tip and by a length 2908 of the tip. The tip 2902 is a portion of the apparatus which is heated and attains high temperatures. In some embodiments the tip 2902 is optionally hollow. In some embodiment the tip base 2910 is optionally hollow. The tip base 2910 serves to attach the tip 2902 to a sheath (not shown).

The description 2904 of the tip 2902 includes a description of an example embodiment which assumes a radius of curvature of the tip of 0.3 millimeter. In some embodiments the tip may not have a shape which is defined by a radius of curvature, and a measure of, by way of a non-limiting example, 0.3 millimeters describes a width or a half-width of the tip. The tip 2902 is also described as having a length of 1 millimeter, and a laser source is also described as having 10 Watts intensity. Using different materials for the tip 2902 results in different times for heating the tip 2902 up to 500 degrees Celsius and cooling the tip 2902 back down to ˜42 degrees Celsius, or approximately body temperature.

In some embodiments using a titanium tip, heating time is optionally approximately 30-100 milliseconds, potentially inversely dependant on thickness of conical envelope, and cooling time is approximately 20 milliseconds.

In an embodiment using a tungsten tip, heating time is approximately 3-15 milliseconds, and cooling time is approximately 3 milliseconds.

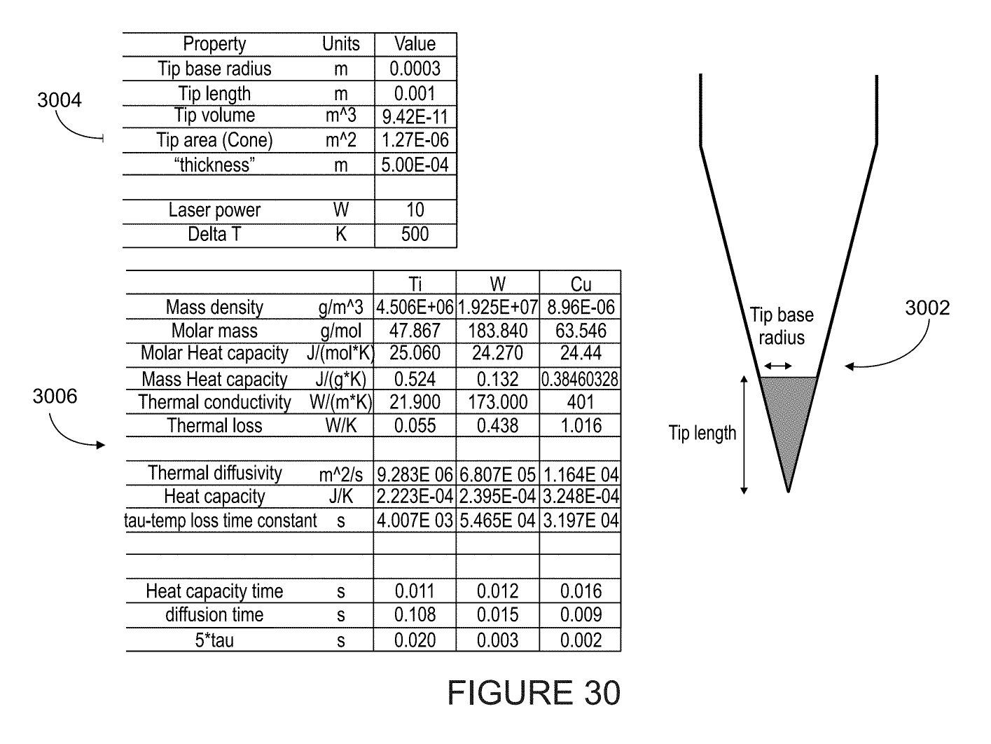

Reference is now made to FIG. 30, which is a simplified illustration of a tip 3002 and two tables 3004 3006 according to an example embodiment of the invention.

The first table 3004 describes units used in describing an example embodiment of a treatment tip and also laser power and a target temperature to which the treatment tip is raised by heating.

The second table 3006 includes units and values describing tips of three different materials, Titanium, Tungsten and Copper. The values in table 3006 include:

Mass density, molar mass, molar heat capacity, mass heat capacity, thermal conductivity, thermal loss, thermal diffusivity, heat capacity, tau-temp loss time constant, heat capacity time, diffusion time and 5*tau. As was shown in FIG. 29, a thermal response time of a treatment tip to being heated by a laser depends on a type of metal used. For a high thermal diffusivity material (see the second table 3006) a thermal response, or relaxation, time, is shorter than for a low thermal diffusivity material. Thermal diffusivity depends on mass density, heat capacity and thermal conduction of the metals, as shown in the second table 3006. Tip dimensions, as seen in the first table 3004, potentially affect both vaporization depth and duration.

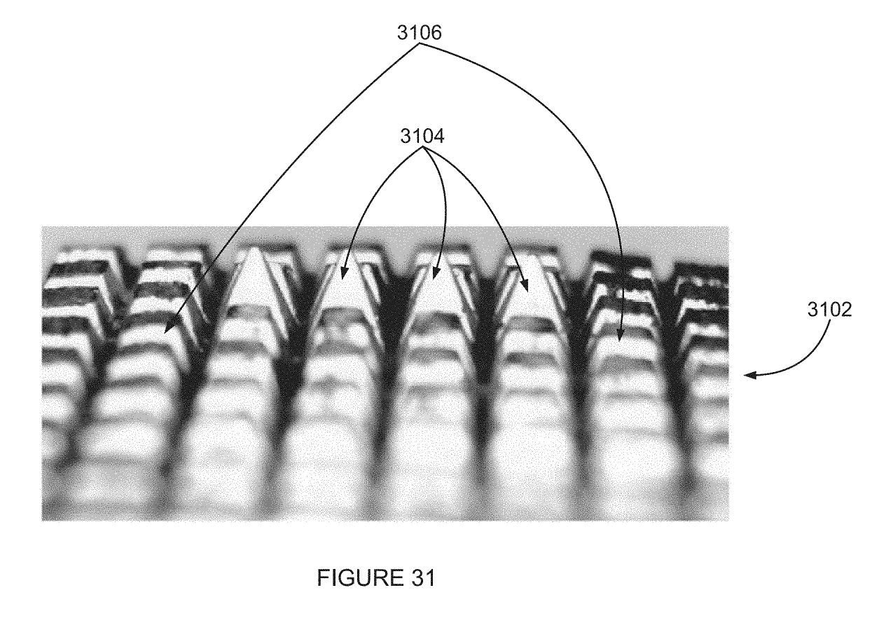

Reference is now made to FIG. 31, which is an image of an array of tips 3102 according to an example embodiment of the invention.

The array of tips 3102 includes some pyramidal tips 3104 and some truncated pyramidal tips 3106. The truncated tips enables application of the sharp tips to be used in surgical incision without contact between other tips and the tissue. The example embodiment depicted in FIG. 31 depicts four sequential sharp tips, however, in some embodiments more or less than four sharp tips, for example in a range of 1-100 sharp tips, and the sharp tips may or may not be sequential.

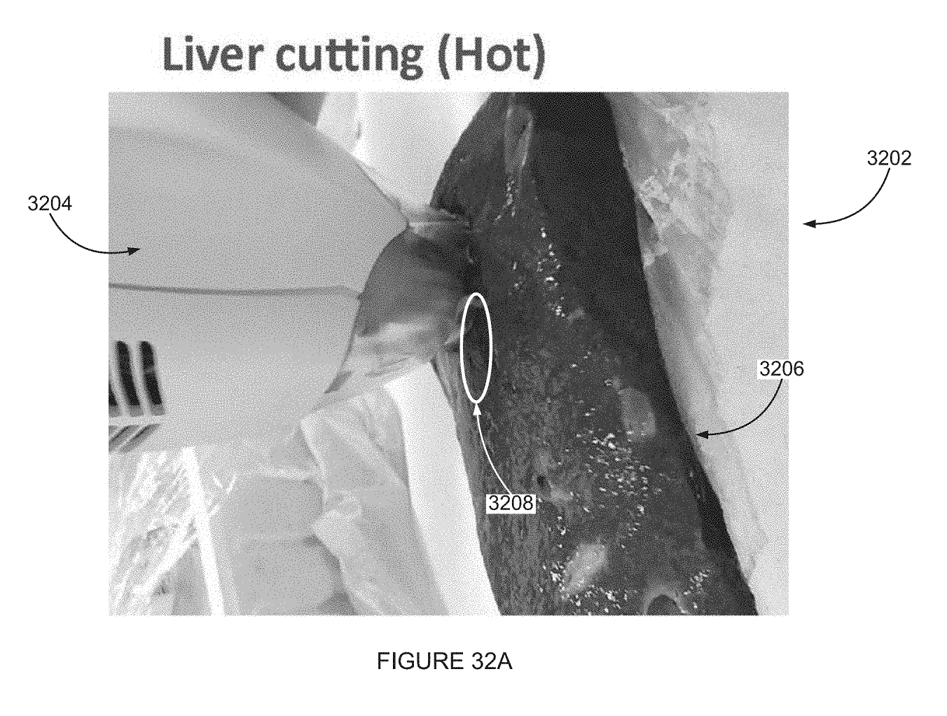

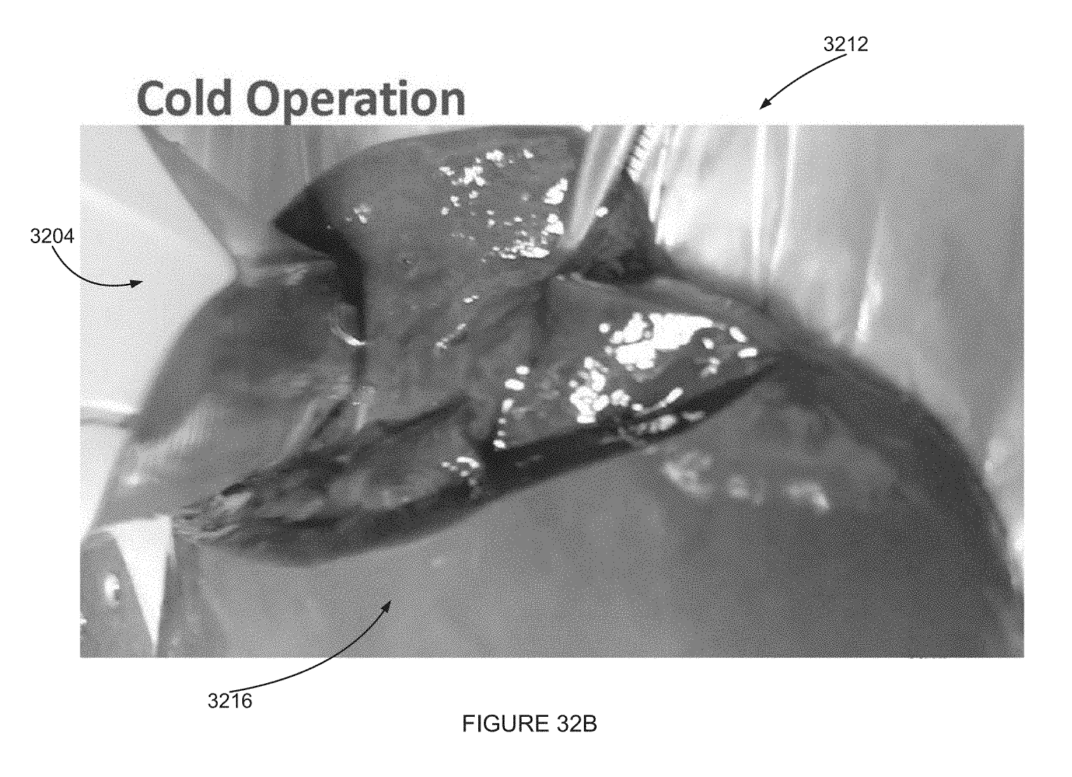

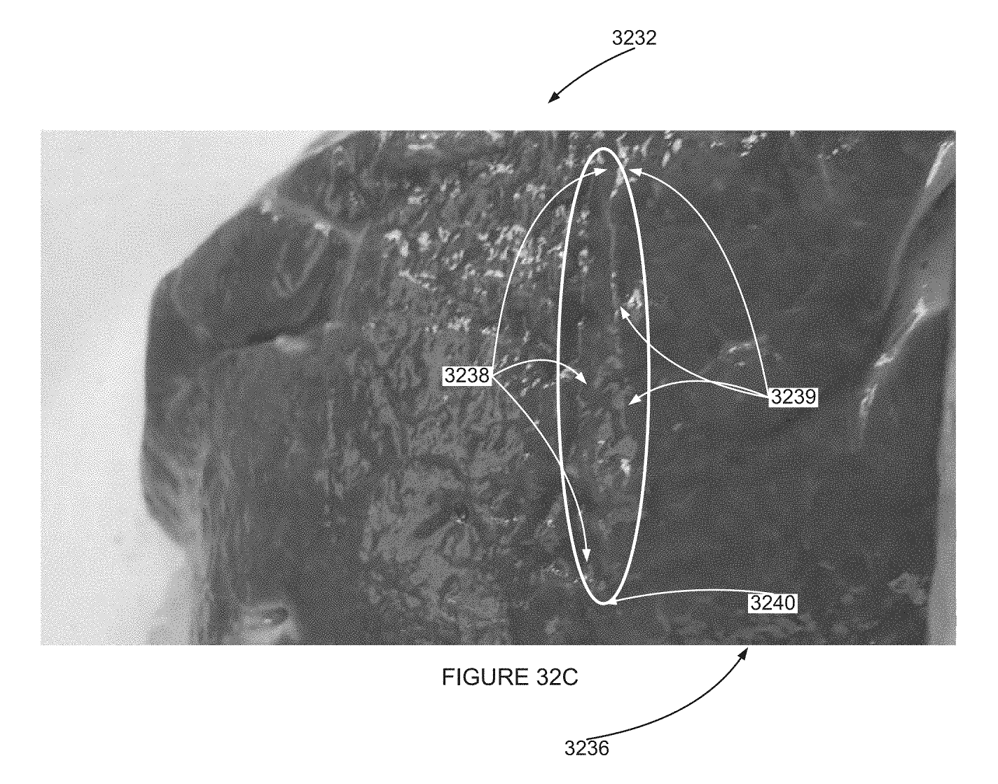

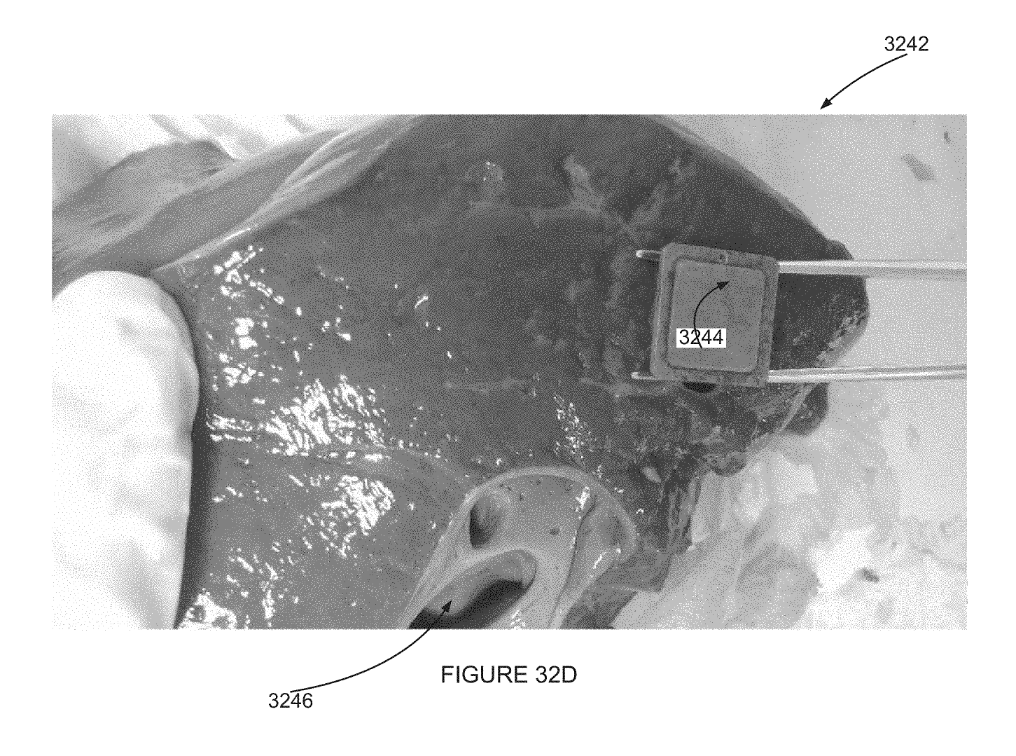

Reference is now made to FIG. 32A, which is an image 3202 of a hand-piece 3204 for thermal surgical vaporization and incision of tissue applied to calf's liver 3206 according to an example embodiment of the invention.

FIG. 32A depicts using the hand-piece 3204 for incising into the calf's liver 3206 using heat, that is, by heating tip(s) in touch with the calf's liver. A linear tip array (not visible in FIG. 32B because of a direction from which the image 3202 was photographed) was held at an approximately constant temperature of 400 deg Celsius and vibrated at a frequency of optionally 13 Hz. The incision formed is difficult to see, and is marked by an ellipse 3208.

Reference is now made to FIG. 32B, which is an image 3212 of the hand-piece 3204 of FIG. 32A applied to calf's liver 3216 according to an example embodiment of the invention. In FIG. 32B the array of tips is cold, or at room temperature. It is noted that no incision has been made to the calf's liver 3216 by the hand-piece 3204, and that appearance of the calf's liver 3216 is not intended to depict any incision. It is noted that in the example embodiment of FIG. 32B the array of tips does not produce an incision when not heated.

Reference is now made to FIG. 32C, which is an image of calf's liver incised with a heated linear tip array according to an example embodiment of the invention.

For producing FIG. 32C, calf liver tissue was pulled aside by a forceps (not shown), in order to avoid contact of the calf liver tissue with an upper portion of the incising tips.

FIG. 32C demonstrates incision quality of an oscillating tip array.

The image 3232 depicts an ellipse 3240 surrounding an incision 3238, and demonstrates a lack of bleeding, with a small coagulation depth and lack of carbonization.

The incision 3238 is visible seen as a vertical incision 3238 in a center of the image 3232. The incision shows no carbonization and there is a very thin (˜50 microns) white coagulation line 3239 on margins of the incision 3238.

Reference is now made to FIG. 32D, which is an image 3242 of an array of tips 3244 of FIG. 32A applied to calf's liver 3246 according to an example embodiment of the invention.

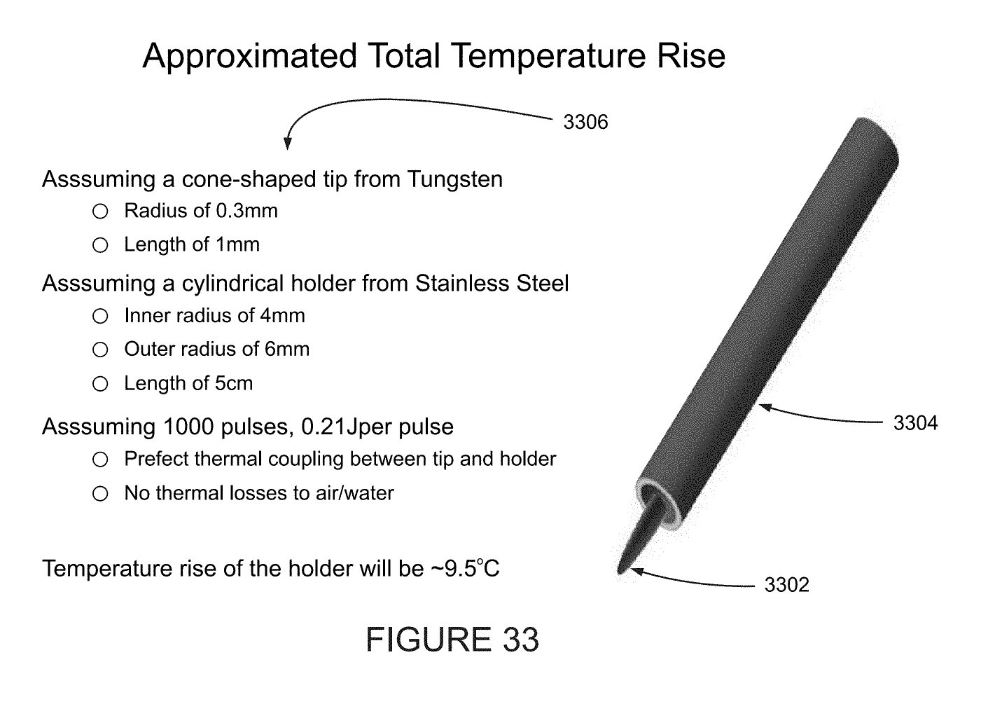

Reference is now made to FIG. 33, which is a simplified illustration of a tip 3302; a tip holder 3304 and a table 3306 according to an example embodiment of the invention.

The tip holder 3304 is attached to the tip 3302.

The table 3306 describes calculation made with relation to heating the tip 3302. The table describes the following:

Assuming a cone-shaped tip 3302 made of Tungsten, having a radius of 0.3 millimeters and a tip length of 1 millimeter; a tip holder 3304 or sheath having an inner radius of 4 millimeters, and outer radius of 6 millimeters and a length of 5 centimeters; applying 1000 pulses of heat of 0.21 Joules per pulse, with perfect thermal coupling between the tip 3302 and the holder 3304 and no additional thermal losses to air or water, a temperature rise of the holder will be approximately 9.5 degrees Celsius.

In some embodiment of the invention the sheath or holder, such as the holder/sheath 3304 depicted in FIG. 33, is optionally chilled actively with air flow or with a water flow.

In some embodiments of the invention the sheath or holder, such as the holder/sheath 3304 depicted in FIG. 33, is not actively chilled. Embodiments which do not use active chilling are suitable when a surgical case lasts just a few minutes, for example between 1-5 minutes, where the holder may be only slightly heated. Such cases are potentially common, since most surgical incisions using an embodiment of the invention are relatively rapid.

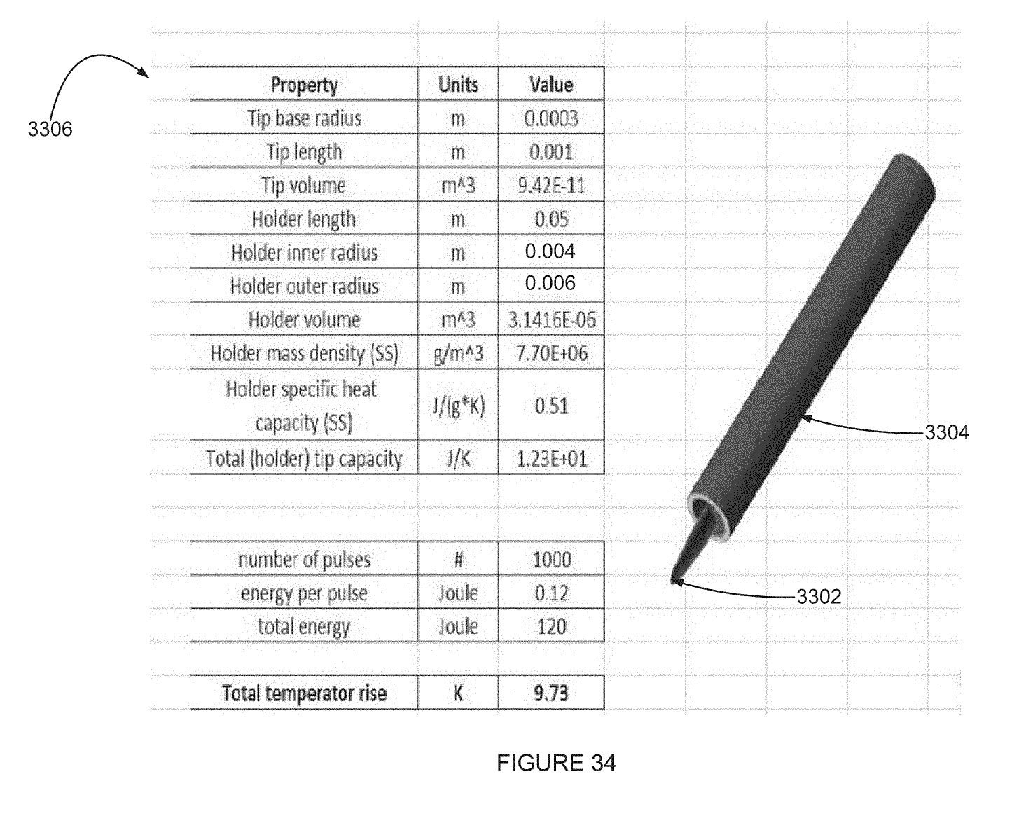

Reference is now made to FIG. 34, which is a simplified illustration of a tip 3402; a tip holder 3404 and a table 3406 according to an example embodiment of the invention.

The tip holder 3404 is attached to the tip 3402.

The table 3406 describes calculation made with relation to heating the tip 3402. The table describes the following:

Assuming a cone-shaped tip 3302 made of tungsten, having a radius of 0.3 millimeters and a tip length of 1 millimeter; a tip volume of 9.42×10⁻¹¹ cubic meters, a tip holder 3304 having a length of 5 centimeters, an inner radius of 4 millimeters, an outer radius of 6 millimeters, a holder material volume of 3.1416 cubic centimeters, a holder mass density of 7.7 grams per cubic centimeter, a holder specific heat capacity of 0.51 Joule/(gram*K), a total holder capacity of 12.3 Joule/K, applying 1000 pulses of heat of 0.12 Joules per pulse, and a total energy applied of 120 Joules—a temperature rise of 9.73 degrees Kelvin is expected in the tip holder. It is noted that typically an operator will not use such a number of pulses without rest, and that typically the temperature rise will not exceed 2-3 degrees Celsius for a sequence of 200-300 pulses.

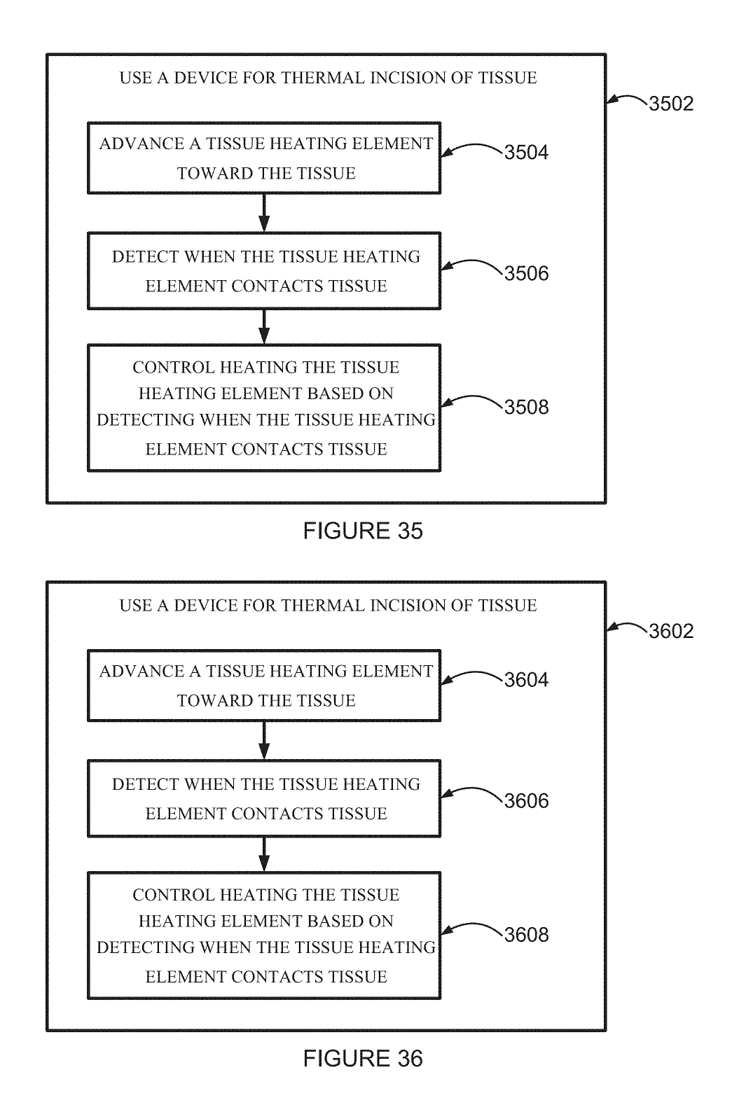

Reference is now made to FIG. 35, which is a simplified flow chart illustration of a method for incising tissue according to an example embodiment of the invention.

The method illustrated in FIG. 35 includes:

using a device for thermal incision of tissue for (3502):

automatically advancing a tissue heating element toward tissue (3504);

automatically detecting when the tissue heating element contacts tissue (3506); and

automatically controlling heating the tissue heating element based on detecting when the tissue heating element contacts tissue (3508).

Reference is now made to FIG. 36, which is a simplified flow chart illustration of a method for introduction of material through a tissue by vaporizing a crater in the tissue according to an example embodiment of the invention.

The method illustrated in FIG. 36 includes:

using a device for thermal incision of tissue for (3602):

automatically advancing a tissue heating element toward tissue (3604);

automatically detecting when the tissue heating element contacts tissue (3606); and

automatically controlling heating the tissue heating element based on detecting when the tissue heating element contacts tissue (3608).

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-2. (canceled)

3. The device according to claim 28 and further comprising:
a laser that heats the tissue heating element; and
an optical fiber that conducts output of the laser to the tissue heating element and
in which the tissue heating element comprises a material opaque to optical energy emitted from the laser.

4. The device according to claim 28 in which the tissue heating element comprises a material selected from a group consisting of:
sapphire;
metal; and
metal coated with a bio-compatible coating.

5-8. (canceled)

9. The device according to claim 28 and further comprising a detector for detecting when the tissue heating element contacts the tissue by measuring mechanical impedance to advancing the tissue heating element.

10. (canceled)

11. The device according to claim 28 in which an oscillatory mechanism that advances the tissue heating element toward tissue and retracts the tissue heating element from tissue comprises an electric motor and the detector that detects when the tissue heating element contacts the tissue comprises a detector that measures current to the electric motor.

12. (canceled)

13. The device according to claim 28 in which the mechanism that advances the tissue heating element toward tissue and retracts the tissue heating element from tissue comprises a linear electric motor.

14. (canceled)

15. The method according to claim 29 and further comprising automatically advancing the tissue heating element a desired distance measured from a point of contact with the tissue.

16. The method according to claim 29 and further comprising automatically retracting the tissue heating element from contact with the tissue.

17-18. (canceled)

19. The method according to claim 29 in which the automatically detecting when the tissue heating element contacts tissue comprises measuring current to an electric motor.

20-21. (canceled)

22. The method according to claim 29 in which heating the tissue heating element is controlled to start only after automatically detecting when the tissue heating element contacts tissue.

23. (canceled)

24. The method according to claim 29 in which heating the tissue heating element is controlled to last for a desired period of time and then stop.

25. The method according to claim 24 in which the desired period of time for heating the tissue is calculated based on an amount of heat required for evaporating a desired volume of tissue.

26-27. (canceled)

28. A device for enabling transfer of material through skin by vaporizing tissue comprising:
a tissue heating element;
a mechanism that advances the tissue heating element toward tissue; and
a heat controller that controls heating and temperature of the tissue heating element,
wherein the mechanism that advances the tissue heating element controls at least one parameter selected from a group consisting of:
duration of contact;
protrusion of the tissue heating element toward the tissue; and
repetition rate of movement of the the tissue heating element toward the tissue,
thereby providing openings in a stratum corneum of the skin for transferring the material therethrough.

29. A method for enabling transfer of material through skin by vaporizing openings in the tissue comprising:
using a device for heating tissue for:
controlling heating and temperature of the tissue heating element;
advancing a tissue heating element toward skin; and
controlling movement of the tissue heating element based on at least one parameter selected from a group consisting of:
duration of contact;
protrusion of the tissue heating element toward the tissue; and
repetition rate of movement of the the tissue heating element toward the tissue,
thereby providing openings in a stratum corneum of the skin for transferring the material therethrough.

30. The method of claim 29 in which a duration of the openings remaining open to introduction of the material, prior to closure by healing, is above 1 hour.

31. The method of claim 29 in which contact time of the tissue heating element with tissue is automatically kept shorter than 10 milliseconds.

32. The method of claim 29 in which a width of a distal end of a tip of the tissue heating element is less than 150 microns.

33. The method of claim 29 in a tip of the tissue heating element comprises a material selected from a group consisting of stainless steel, tungsten, nickel, copper, gold and titanium.

34. The method of claim 30 in which the duration is above 6 hours.

35. The method according to claim 29 and further comprising automatically detecting when the tissue heating element contacts tissue by measuring mechanical impedance to advancing the tissue heating element.
